(19) United States
(12) Patent Application Publication
(10) Pub. No.: US 2024/0043824 A1
(43) Pub. Date: Feb. 8, 2024

(54) MULTIMERIC PROTEINS OF THE PEROXIREDOXIN FAMILY AS SCAFFOLD PROTEINS

(71) Applicants: UNIVERSITE DE LORRAINE, Nancy (FR); CENTRA NATIONAL DE LA RECHERCHE SCIENTIFIQUE - CNRS -, Paris (FR); INSTITUT NATIONAL DE LA SANTÉ ET DE LA RECHERCHE MÉDICALE, Paris (FR)

(72) Inventors: Marc QUINTERNET, Chaligny (FR); Alexandre KRIZNIK, Villers Les Nancy (FR); Marie-Eve CHAGOT, Nancy (FR)

(21) Appl. No.: 18/266,109

(22) PCT Filed: Dec. 10, 2021
(86) PCT No.: PCT/EP2021/085149

§ 371 Date: Jun. 8, 2023

(30) Foreign Application Priority Data
Dec. 10, 2020 (EP) 20306539.6

(51) Int. Cl. C12N 11/06; C12N 9/08; C12N 15/70

(52) U.S. Cl. CPC C12N 11/06; C12N 9/0065; C12Y 111/01015; C12N 15/70

(57) ABSTRACT

The present invention relates to the use of a multimeric protein of the peroxiredoxin family as a scaffold protein, characterized in that one or more proteins(s) of interest are linked to one or two N- and C-terminal end(s) of one or more monomers(s) of the peroxiredoxin, said peroxiredoxin having no redox activity.

### TECHNICAL FIELD

The present invention relates to the use of a multimeric protein from the family of the peroxiredoxins (Prxs) as a scaffold protein, characterized in that one or more proteins of interest are linked to one or two N- and C-terminal end(s) of one or more monomers of said peroxiredoxin, said peroxiredoxin having no redox activity.

### PRIOR ART

Enzymes are biocatalysts which are capable of accelerating the synthesis, the modification or the breakdown of molecules, with the particular feature of working optimally under conditions which are compatible with the living body. They are employed in numerous fields such as bioplastics, biofuels or the synthesis of therapeutic molecules.

Numerous studies have been conducted for modifying the catalytic power of enzymes. The rapid expansion of genetic engineering has therefore enabled activating mutations to be introduced on certain enzymes. The advances in three-dimensional protein structure resolution and in bioinformatics have enabled these mutations to be guided in a rational manner (Goldenzweig et al., 2016, Mol Cell. 2016 Jul. 21; 63(2):337-346).

Another approach involves the local concentration of reactants, intermediates and enzymes by assembling them in the same complex so as to improve the efficacy of the biochemical reactions. Various methods allowing artificial assembly of the enzymes have been developed. For example, the direct fusion of enzymes with one another has been used to coordinate the expression and the localization of two enzymes in the biosynthesis of resveratrol in such a way as to augment the production of the product in the cells (Zhang et al., J. Am. Chem. Soc. 128:13030-13031 (2006)). Nevertheless, the structure of the two enzymes fused together may be adversely affected and bring about deactivation of the enzymes.

Another approach is to immobilize the enzymes on a proteinic support called a scaffold protein. The scaffold protein will enable the local concentration of one or more enzymes in the vicinity of a substrate of interest, this being particularly suitable for the catalysis of polymeric substrates, or will enable enzymes belonging to the same metabolic pathway to be brought spatially close, so accelerating and simplifying the synthesis of small molecules (Morais et al., 2010; Horn et al., 2015).

An artificial multiprotein complex derived from the cellulosome, in which the enzymes have been integrated at specific sites, has been used to improve the breakdown of cellulose (Fierobe et al. 2001. J. Biol. Chem. 276:21257-21261 and Fierobe et al. 2005, J Biol Chem. 280(16):16325-34). Scaffold proteins which utilize protein-protein interaction domains have also been developed for improving the efficacy of production of molecules of interest such as mevalonate or glucaric acid (Dueber et al. 2009. Nat. Biotechnol. 27:753-759).

There remains, nevertheless, a need to develop new scaffold proteins which are strongly expressed in the cells while remaining stable under diverse environmental conditions.

Peroxiredoxins (Prxs) are proteins which are found throughout the kingdom of the living organism, including extremophilic organisms. Peroxiredoxins are resistant to highly diverse conditions of pH, of temperature and of saline concentrations. They have acquired a high stability so as to ensure their redox functions, which are indispensable to the continued existence of the cells. Their sequences include a common motif which is organized around cysteines that are important for the functioning. The variety of the sequences surrounding this motif endows the host organism with appropriate physicochemical properties. This protein family shares a common architecture which is based on a monomer of approximately 20 kDa that is capable of multimerization to form, in the majority of cases, a decameric or dodecameric ring. By virtue of this specific three-dimensional arrangement, Prxs such as TSA1 from S. cerevisiae exhibit floating ends situated at the N- and C-terminal positions of the monomers.

### SUMMARY OF THE INVENTION

The inventors have made use of the properties of multimeric proteins from the family of the peroxiredoxins, the TSA1 protein from Saccharomyces cerevisiae or the peroxiredoxin from Pyrococcus furiosus, in order to develop scaffold proteins capable of being used in diverse environmental conditions. To suppress the intrinsic redox activity of the Prxs and to retain only the support function of the Prx, the active cysteines have been mutated. The inventors have shown that the decameric structure of the Prx remains conserved, in spite of the linking of the proteins of interest in N- and/or C-terminal positions of the Prxs monomers, and that the function of the protein of interest is maintained, making the peroxiredoxin a scaffold protein having particular properties.

The present invention relates to the use of a multimeric protein from the family of the peroxiredoxins as a scaffold protein, characterized in that one or more proteins of interest are linked to one or two N- and C-terminal end(s) of one or more monomers of said peroxiredoxin, said peroxiredoxin having no redox activity. In one particular embodiment, said one or more proteins of interest are enzymes and the scaffold protein is used for augmenting the catalytic activity of the enzymes, or said proteins of interest are proteins of the same signaling pathway and the scaffold protein is used for augmenting the efficacy of signaling.

The peroxiredoxin is preferably the peroxiredoxin TSA1 from Saccharomyces cerevisiae of SEQ ID NO: 1 and comprises more particularly a cysteine mutated in position 48 and/or in position 171 of the sequence SEQ ID NO: 1, preferably each substituted by an alanine or a serine.

In another preferred embodiment, the peroxiredoxin is the peroxiredoxin from Pyrococcus furiosus of SEQ ID NO: 16 and comprises more particularly a cysteine mutated in position 46 and/or in position 211 of the sequence SEQ ID NO: 16, preferably each substituted by an alanine or a serine.

In one particular embodiment, the protein of interest is fused to one or two N- and C-terminal end(s) of said peroxiredoxin monomer via a linking sequence.

In another particular embodiment, said peroxiredoxin monomer and the protein of interest are linked via an adapter/peptide ligand pair.

According to another aspect, the invention relates to a multimeric protein from the family of the peroxiredoxins, comprising at least one protein of interest linked to one or two N- and C-terminal end(s) of one or more peroxiredoxin monomers having no redox activity, characterized in that said peroxiredoxin monomer and the protein of interest are linked via an adapter/peptide ligand pair. The invention also relates to a multimeric protein from the family of the peroxiredoxins comprising at least two different proteins of interest which are linked to one or two N- and C-terminal end(s) of one or more peroxiredoxin monomers having no redox activity. The peroxiredoxin is preferably the peroxiredoxin TSA1 from Saccharomyces cerevisiae of SEQ ID NO: 1, more particularly comprising a cysteine mutated in position 48 and/or in position 171 of SEQ ID NO: 1, preferably each substituted by an alanine or a serine.

In another preferred embodiment, the peroxiredoxin is the peroxiredoxin from Pyrococcus furiosus of SEQ ID NO: 16 and comprises more particularly a cysteine mutated in position 46 and/or in position 211 of the sequence SEQ ID NO: 16, preferably each substituted by an alanine or a serine.

In one preferred embodiment, said peroxiredoxin monomer and the protein of interest are linked via an adapter/peptide ligand pair.

The invention additionally relates to one or more nucleotide constructions encoding for a multimeric protein as described above and to an expression vector comprising said nucleotide construction. The invention also relates to a host cell comprising a multimeric protein, one or more nucleotide constructions or an expression vector, all as described above.

### FIGURE LEGENDS

FIG. 1: (A) From left to right, SDS-PAGE analysis of the covalent grafts TSA1-CRD_{SAT}, CRD_{SAT}-TSA1 and CRD_{SAT}-TSA1-(GGGS)3-CRD_{SAT}. The proteins of interest, indicated by an arrow, are recovered in the sonication supernatant (SSo), on the Lactose-Sepharose resin (Lac-Seph) and in the elution fractions (Elutions 1 to 3). (B) Size exclusion chromatography profiles obtained on the three constructions indicated in (A). (C) the fractions corresponding to the main peaks (P1 and P2) obtained in (B) are analyzed by SDS-PAGE. (D) Analysis by dynamic light scattering of the peaks P1 obtained in (B) and of the ungrafted protein TSA1. The average size in nanometers is reported.

FIG. 2: Analysis by SDS-PAGE of the reconstitution of a complex of 6×HIS-SPAG1-TPR3 with CRD_{SAT}-TSA1-(GGGS)_{2 or 3}-HSP90pep. SSo and TALON denote, respectively, the sonication supernatants and the affinity resin.

FIG. 3: DLS spectra (DLS: dynamic light scattering) of free Pfu-PRX (A), of the scaffolded PreScission 3C and of the scaffolded PETase (C). The size of each of the majority species is indicated.

FIG. 4: Thermograms recorded on Tsa1 (gray) and Pfu-Prx (black) obtained by isothermal titration microcalorimetry at 25° C.

FIG. 5: Denaturation curve obtained for Pfu-Prx by differential scanning microcalorimetry. The half-denaturation temperature measured at the summit of each peak is indicated.

FIG. 6: Monitoring of the breakdown of 6His-SPAG1-TPR3 by the protease 3C scaffolded on Pfu-Prx by electrophoresis on SDS denaturing gel. The lane labeled “Pfu-Prx:3C” corresponds to the scaffolded protease. The lane labeled “substrate” corresponds to 6His-SPAG1-Cter after 60 minutes at 10° C. in the absence of protease. The lanes labeled 1 to 60 correspond to the deposition of samples of reaction medium taken after 1, 2, 5, 10, 15, 30 and 60 min. The lane labeled “MT” corresponds to the size marker (MT) expressed in kDa.

FIG. 7: Monitoring of the pH of the reaction medium during the breakdown of PET by the PETase scaffolded on Pfu-Prx. The black curve corresponds to the PET in the presence of enzyme. The gray curve corresponds to the control without enzyme.

### DETAILED DESCRIPTION OF THE INVENTION

The inventors have shown that the multimeric protein of peroxiredoxin in which the free N- and C-terminal ends of the monomers are linked to a protein of interest retains its particular three-dimensional structure as decamer or dodecamer and maintains the function of the protein of interest. The multimeric protein of the peroxiredoxin may therefore be used as a scaffold protein.

The terms “peptide”, “polypeptide” and “protein” are used interchangeably and correspond to a chain of amino acids linked by peptide linkages, irrespective of the number of amino acids forming this chain.

A scaffold protein is a protein which enables the linking of multiple, identical or different proteins of interest in a single proteinic complex in order to facilitate interactions between the various proteins of interest and their functions or to assemble the proteins of interest locally.

The scaffold protein, by linking multiple proteins of interest, makes it possible, for example, to assemble compounds of the same metabolic pathway in a single complex in order to amplify the efficacy of the enzymatic reactions by limiting those interactions which are unnecessary and by augmenting the proximity and concentration of the proteins of interest. The scaffold protein may also allow proteins of interest to be localized in a specific zone of the cell in order to improve the function of the protein of interest.

The peroxiredoxin proteins (Prx or PRDX) (EC 1.11.1.24), which are also called TSA (Thiol-specific antioxidants), are highly conserved enzymes with peroxidase activity which is dependent on a cysteine residue called Peroxidatic cysteine (Cp). Peroxiredoxins reduce peroxides via their oxidation site. The Cp cysteine subsequently reacts with a “resolving” cysteine (C_{R}) to form a disulfide bridge, which is reduced by an electron donor to complete a catalytic cycle (Revue Rhee SG, Mol Cells. 2016. 39(1):1-5).

Peroxiredoxins may be classified in subclasses 2-Cys, atypical 2-Cys and 1-Cys peroxiredoxin according to whether the latter has or does not have a C_{R} cysteine. In the atypical 2-Cys subclass of the Prxs, the C_{R} cysteine is located in a nontypical position.

More recently, peroxiredoxins have also been classified in six classes, called AhpC-Prx1, BCP-PrxQ, Prx5, Prx6, Tpx and AhpE (Soito, Laura et al. 2011, Nucleic Acids Res. England. 39 (Database issue): D332-7. doi:10.1093/nar/gkq1060).

The members of the subclass AhpC/Prx1 and Prx6 associate with one another to form octamers, decamers or else dodecamers (Hall et al. 2010, J Mol Biol. 402(1):194-209; Parsonage et al. 2005, Biochemistry, 2005; 44(31):10583-92).

The peroxiredoxins of the present patent application are peroxiredoxin monomers which are capable of undergoing multimerization, preferably to form decamer or dodecamer, more particularly of the subclass of the AhpC-Prx1 or Prx6. The Prx monomers are preferably from the subclass of the AhpC-Prx1.

The subclass AhpC-Prx1 is essentially composed of the “typical 2-Cys” Prxs. The members of this subclass comprise bacterial AhpC proteins, tryparedoxin peroxidases, plant Prxs, including the 2-Cys Prxs from Arabidopsis thaliana and the Bas1 Pxrs from barley, the yeast proteins TSA1 and TSA2, and human Prx1, II, III and IV. In addition to the common base structure of the Prxs, the members of this subclass have a C-terminal extension which contains the C_{R}, which forms a disulfide linkage with the Cp in its partner subunit through the type-B interface (Hall et al. 2010, J Mol Biol. 402(1):194-209).

The subclass Prx6 comprises the bacterial Prx6 proteins, the 1-Cys plant Prxs of Arabidopsis thaliana and of barley, the mitochondrial yeast protein Prx1 and human PrxVI. The Prx6 proteins are close to the AhpC/Prx1 subclass. The Prx6 proteins comprise a C-terminal extension and form type-B dimers and, in certain cases, higher oligomeric states. However, in contrast to the AhpC-Prx1 subclass, the members of the Prx6 subclass are primarily 1-Cys, although there are 2-Cys representatives in existence (Deponte and Becker 2005).

TABLE 1
Sequences of peroxiredoxin monomers capable of multimerization. The
peroxidatic cysteine is highlighted in gray, and the “resolving” cysteine is indicated in bold
and underlined.

| | | | | | SEQ |
|---|---|---|---|---|---|
| | | Uniprot | | | ID |
| Protein name | Organism | ID | Folding | Sequence | NO |
| TSA1 | Saccharomyces | P34760 | 10- | MVAQVQKQAPTFKKTAVVDGVFDEVSLDKY | 1 |
| | cerevisiae | | mer | KGKYVVLAFIPLAFTFVCPTEIIAFSEAAK | |
| | | | | KFEEQGAQVLFASTDSEYSLLAWTNIPRKEGG | |
| | | | | LGPINIPLLADTNHSLSRDYGVLIEEEG | |
| | | | | VALRGLFIIDPKGVIRHITINDLPVGRNVDEALR | |
| | | | | LVEAFQWTDKNGTVLPCNWTPGAATI | |
| | | | | KPTVEDSKEYFEAANK | |
| Prx APK1 | Aeropyrum | Q9Y9L0 | 10- | MPGSIPLIGERFPEMEVTTDHGVIKLPDHYVS | 2 |
| | pernix K1 | | mer | QGKWFVLFSHPADFTPVCTTEFVSFARR | |
| | | | | YEDFQRLGVDLIGLSVDSVFSHIKWKEWIERHI | |
| | | | | GVRIPFPIIADPQGTVARRLGLLHAES | |
| | | | | ATHTVRGVFIVDARGVIRTMLYYPMELGRLVD | |
| | | | | EILRIVKALKLGDSLKRAVPADWPNNEI | |
| | | | | IGEGLIVPPPTTEDQARARMESGQYRCLDWW | |
| | | | | FCWDTPASRDDVEEARRYLRRAAEKPAKL | |
| | | | | LYEEARTHLH | |
| Tryparedoxin | Crithidia | Q9TZX2 | 10- | MSCGAAKLNHPAPEFDDMALMPNGTFKKVS | 3 |
| peroxidase CF | fasciculata | | mer | LSSYKGKYVVLFFYPMDFTFVCPTEIIQFS | |
| | | | | DDAKRFAEINTEVISCSCDSEYSHLQWTSVDR | |
| | | | | KKGGLGPMAIPMLADKTKAIARAYGVLD | |
| | | | | EDSGVAYRGVFIIDPNGKLRQIIINDMPIGRNV | |
| | | | | EEVIRLVEALQFVEEHGEVCPANWKKG | |
| | | | | DAKKKEGH | |
| Prx CR | Chlamydomonas | Q9FE86 | 10- | MAALQSASRSSAVAFSRQARVAPRVAASVAR | 4 |
| | reinhardtii | | mer | RSLVVRASHAEKPLVGSVAPDFKAQAVFD | |
| | | | | QEFQEITLSKYRGKYVVLFFYPLDFTFVCPTEIT | |
| | | | | AFSDRYKEFKDINTEVLGVSVDSQFT | |
| | | | | HLAWIQTDRKEGGLGDLAYPLVADLKKEISKA | |
| | | | | YGVLTEDGISLRGLFIIDKEGVVQHATI | |
| | | | | NNLAFGRSVDETKRVLQAIQYVQSNPDEVCP | |
| | | | | AGWKPGDKTMKPDPKGSKEYFSAV | |
| 2-Cys | Arabidopsis | Q96291 | 10- | MASVASSTTLISSPSSRVFPAKSSLSSPSVSFLRT | 5 |
| peroxiredoxin | thaliana | | mer | LSSPSASASLRSGFARRSSLSSTSR | |
| BAS1, | | | | RSFAVKAQADDLPLVGNKAPDFEAEAVFDQE | |
| chloroplastic | | | | FIKVKLSDYIGKKYVILFFYPLDFTFVCP | |
| | | | | TEITAFSDRHSEFEKLNTEVLGVSVDSVFSHLA | |
| | | | | WVQTDRKSGGLGDLNYPLISDVTKSIS | |
| | | | | KSFGVLIHDQGIALRGLFIIDKEGVIQHSTINNL | |
| | | | | GIGRSVDETMRTLQALQYIQENPDEV | |
| | | | | CPAGWKPGEKSMKPDPKLSKEYFSAI | |
| Mitochondrial | Leishmania | Q95U89 | 10- | MLRRLPTSCFLKRSQFRGFAATSPLLNLDYQM | 6 |
| Prx LI | infantum | | mer | YRTATVREAAPQFSGQAVVNGAIKDINM | |
| | | | | NDYKGKYIVLFFYPMDFTFVCPTEIIAFSDRHA | |
| | | | | DFEKLNTQVVAVSCDSVYSHLAWVNTP | |
| | | | | RKKGGLGEMHIPVLADKSMEIARDYGVLIEES | |
| | | | | GIALRGLFIIDKKGILRHSTINDLPVGR | |
| | | | | NVDEALRVLEAFQYADENGDAIPCGWKPGQ | |
| | | | | PTLDTTKAGEFFEKNM | |
| Prx1 RN | Rattus | Q63716 | 10- | MSSGNAKIGHPAPSFKATAVMPDGQFKDISL | 7 |
| | norvegicus | | mer | SDYKGKYVVFFFYPLDFTFVCPTEIIAFS | |
| | | | | DRAEEFKKLNCQVIGASVDSHFCHLAWINTPK | |
| | | | | KQGGLGPMNIPLVSDPKRTIAQDYGVLK | |
| | | | | ADEGISFRGLFIIDDKGILRQITINDLPVGRSVD | |
| | | | | EILRLVQAFQFTDKHGEVCPAGWKPG | |
| | | | | SDTIKPDVNKSKEYFSKQK | |
| Tryparedoxin | Leishmania | Q4QF76 | 10- | MSCGNAKINSPAPSFEEVALMPNGSFKKISLSS | 8 |
| peroxidase LM | major | | mer | YKGKWVVLFFYPLDFTFVCPTEVIAFS | |
| | | | | DSVSRFNELNCEVLACSIDSEYAHLQWTLQDR | |
| | | | | KKGGLGTMAIPMLADKTKSIARSYGVLE | |
| | | | | ESQGVAYRGLFIIDPHGMLRQITVNDMPVGR | |
| | | | | SVEEVLRLLEAFQFVEKHGEVCPANWKKG | |
| | | | | APTMKPEPNASVEGYFSKQ | |
| Prx-4 (isoform | Homo sapiens | Q13162 | 10- | MEALPLLAATTPDHGRHRRLLLLPLLLFLLPAG | 9 |
| 1) HS | | | mer | AVQGWETEERPRTREEECHFYAGGQVY | |
| | | | | PGEASRVSVADHSLHLSKAKISKPAPYWEGTA | |
| | | | | VIDGEFKELKLTDYRGKYLVFFFYPLDF | |
| | | | | TFVCPTEIIAFGDRLEEFRSINTEVVACSVDSQF | |
| | | | | THLAWINTPRRQGGLGPIRIPLLSDL | |
| | | | | THQISKDYGVYLEDSGHTLRGLFIIDDKGILRQI | |
| | | | | TLNDLPVGRSVDETLRLVQAFQYTDK | |
| | | | | HGEVCPAGWKPGSETIIPDPAGKLKYFDKLN | |
| TSA2 | Saccharomyces | Q04120 | 10- | MVAEVQKQAPPFKKTAVVDGIFEEISLEKYKG | 10 |
| | cerevisiae | | mer | KYVVLAFVPLAFSFVCPTEIVAFSDAAK | |
| | | | | KFEDQGAQVLFASTDSEYSLLAWTNLPRKDG | |
| | | | | GLGPVKVPLLADKNHSLSRDYGVLIEKEG | |
| | | | | IALRGLFIIDPKGIIRHITINDLSVGRNVNEALRL | |
| | | | | VEGFQWTDKNGTVLPCNWTPGAATI | |
| | | | | KPDVKDSKEYFKNANN | |
| Alkyl | Helicobacter | P56876 | 10- | MLVTKLAPDFKAPAVLGNNEVDEHFELSKNL | 11 |
| hydroperoxide | pylori | | mer | GKNGVILFFWPKDFTFVCPTEIIAFDKRV | |
| reductase C HP | | | | KDFHEKGFNVIGVSIDSEQVHFAWKNTPVEK | |
| | | | | GGIGQVSFPMVADITKSISRDYDVLFEEA | |
| | | | | IALRGAFLIDKNMKVRHAVINDLPLGRNADE | |
| | | | | MLRMVDALLHFEEHGEVCPAGWRKGDKGM | |
| | | | | KATHQGVAEYLKENSIKL | |
| Prx-2 (isoform | Homo sapiens | P32119 | 10- | MASGNARIGKPAPDFKATAVVDGAFKEVKLS | 12 |
| 1) HS | | | mer | DYKGKYVVLFFYPLDFTFVCPTEIIAFSN | |
| | | | | RAEDFRKLGCEVLGVSVDSQFTHLAWINTPRK | |
| | | | | EGGLGPLNIPLLADVTRRLSEDYGVLKT | |
| | | | | DEGIAYRGLFIIDGKGVLRQITVNDLPVGRSVD | |
| | | | | EALRLVQAFQYTDEHGEVCPAGWKPGS | |
| | | | | DTIKPNVDDSKEYFSKHN | |
| Alkyl | Salmonella | P0A251 | 10- | MSLINTKIKPFKNQAFKNGEFIEVTEKDTEGR | 13 |
| hydroperoxide | typhimurium | | mer | WSVFFFYPADFTFVCPTELGDVADHYEE | |
| reductase C ST | | | | LQKLGVDVYSVSTDTHFTHKAWHSSSETIAKIK | |
| | | | | YAMIGDPTGALTRNFDNMREDEGLADR | |
| | | | | ATFVVDPQGIIQAIEVTAEGIGRDASDLLRKIKA | |
| | | | | AQYVAAHPGEVCPAKWKEGEATLAPS | |
| | | | | LDLVGKI | |
| Thioredoxin | Schistosoma | O97161 | 10- | MVLLPNRPAPEFKGQAVINGEFKEICLKDYRG | 14 |
| peroxidase SM | mansoni | | mer | KYVVLFFYPADFTFVCPTEIIAFSDQVE | |
| | (Blood fluke) | | | EFNSRNCQVIACSTDSQYSHLAWDNLDRKSG | |
| | | | | GLGHMKIPLLADRKQEISKAYGVFDEEDG | |
| | | | | NAFRGLFIIDPNGILRQITINDKPVGRSVDETLR | |
| | | | | LLDAFQFVEKHGEVCPVNWKRGQHGI | |
| | | | | KVNQK | |
| Tryparedoxin | Trypanosoma | O96763 | 10- | MSCGDAKLNHPAPDFNETALMPNGTFKKVA | 15 |
| peroxidaseTC | cruzi | | mer | LTSYKGKWLVLFFYPMDFTFVCPTEICQFS | |
| | | | | DRVKEFSDIGCEVLACSMDSEYSHLAWTSIER | |
| | | | | KRGGLGQMNIPILADKTKCIMKSYGVLK | |
| | | | | EEDGVAYRGLFIIDPKQNLRQITVNDLPVGRD | |
| | | | | VDEALRLVKAFQFVEKHGEVCPANWKPG | |
| | | | | DKTMKPDPEKSKEYFGAVA | |
| Prx PF | Pyrococcus | Q8U218 | 10- | MIVIGEKFPEVEVKTTHGVIKLPDHFTKQGKW | 16 |
| | furiosus | | mer | FMLFSHPADFTPVCTTEFYGLQIRLEKFRELGV | |
| | | | | EPIGLSVDQVFSHLKWMEWIKEKLGVEIEFPVI | |
| | | | | ADDRGDLAEKLGMIPSGSTITARAVFIVDDKGI | |
| | | | | IRAIVYYPAEVGRDWDEILRLVKALKISTEKGVA | |
| | | | | LPHKWPNNELIGDKVIVPPASSVEQIKEREEAK | |
| | | | | AKGEIECYDWWFCYKKLE | |
| Prx PH | Pyrococcus | 058966 | 10- | MVVIGEKFPEVEVKTTHGVIKLPDYFTKQGKW | 17 |
| | horikoshii | | mer | FILFSHPADFTPVCTTEFYGMQKRVEEF | |
| | | | | RKLGVEPIGLSVDQVFSHIKWIEWIKDNLSVEI | |
| | | | | DFPVIADDRGELAEKLGMIPSGATITA | |
| | | | | RAVFVVDDKGIIRAIVYYPAEVGRDWDEILRLV | |
| | | | | KALKISTEKGVALPHKWPNNELIGDKV | |
| | | | | IVPPASTIEEKKQREEAKAKGEIECYDWWFCYK | |
| | | | | KLE | |
| Prx-4 MM | Mus musculus | O08807 | 10- | MEARSKLLDGTTASRRWTRKLVLLLPPLLLFLL | 18 |
| | | | mer | RTESLQGLESDERFRTRENECHFYAGG | |
| | | | | QVYPGEASRVSVADHSLHLSKAKISKPAPYWE | |
| | | | | GTAVINGEFKELKLTDYRGKYLVFFFYP | |
| | | | | LDFTFVCPTEIIAFGDRIEEFKSINTEVVACSVDS | |
| | | | | QFTHLAWINTPRRQGGLGPIRIPLL | |
| | | | | SDLNHQISKDYGVYLEDSGHTLRGLFIIDDKGV | |
| | | | | LRQITLNDLPVGRSVDETLRLVQAFQY | |
| | | | | TDKHGEVCPAGWKPGSETIIPDPAGKLKYFDK | |
| | | | | LN | |
| Alkyl | Amphibacillus | K0J4Q8 | 10- | MSLIGTEVQPFRAQAFQSGKDFFEVTEADLKG | 19 |
| hydroperoxide | xylanus | | mer | KWSIVVFYPADFSFVCPTELEDVQKEYA | |
| reductase C AX | | | | ELKKLGVEVYSVSTDTHFVHKAWHENSPAVG | |
| | | | | SIEYIMIGDPSQTISRQFDVLNEETGLAD | |
| | | | | RGTFIIDPDGVIQAIEINADGIGRDASTLINKVK | |
| | | | | AAQYVRENPGEVCPAKWEEGGETLKP | |
| | | | | SLDIVGKI | |
| Prx-1 AC | Ancylostoma | J7HJM3 | 10- | MSKAFIGKPAPDFATKAVFDGDFVDVKLSDYK | 20 |
| | ceylanicum | | mer | GKYVVLFFYPLDFTFVCPTEIIAFSDRF | |
| | | | | PEFKNLNVAVLACSTDSVFSHLAWINTPRKHG | |
| | | | | GLGDMKIPVLADTNHQIAKDYGVLKDDE | |
| | | | | GIAYRGLFIIDPKGILRQITINDLPVGRSVDETLR | |
| | | | | LVQAFQYTDKHGEVCPAGWTPGKDT | |
| | | | | IKPAVKESKEYFNKAN | |
| Alkyl | Enterococcus | H7C7A0 | 10- | MNLINQKLFDFECDAYHDGEFTRVSTEDILGK | 21 |
| hydroperoxide | faecalis | | mer | WSIFFFYPADFSFVCPTELGDMQEHYAH | |
| reductase C EF | | | | LQELNCEVYSVSEDSHYVHKAWADATETIGKI | |
| | | | | KYPMLADPNGQLARFFGVLDEASGMAYR | |
| | | | | ASFIVSPEGDIKSYEINDMGIGRNAEELVRKLE | |
| | | | | ASQFVAEHGDKVCPANWQPGEETIAPS | |
| | | | | LDLVGKI | |
| Prx-4 LC | Larimichthys | H3JQV8 | 10- | MEEAAHVKNSQCHNYAGGHVYPGEAFRVPV | 22 |
| | crocea | | mer | SDHSLHLSKAKISKPAPQWEGTAVINGEFK | |
| | | | | ELKLSDYRGKYLVFFFYPLDFTFVCPTEIIAFSDR | |
| | | | | VHEFRAINTEVVACSVDSQFTHLAW | |
| | | | | IITPRKQGGLGPMKIPLLSDLTHQISKDYGVYLE | |
| | | | | DQGHTLRGLFIIDEKGVLRQITMNDL | |
| | | | | PVGRSVDETLRLVQAFQYTDKHGEVCPAGWK | |
| | | | | PGSDTIIPDPSGKLKYFDKMK | |
| Prx SI | Sulfolobus | F0NEA3 | 10- | MSEGRIPLIGEKFPEMEVITTHGKIKLPDDYKG | 23 |
| | islandicus | | mer | RWFVLFSHPGDFTPVCTTEFYSFSKKY | |
| | | | | EEFKKLNTELIGLSVDSNISHIEWVMWIEKNLK | |
| | | | | VEVPFPIIADPMGNVAKRLGMIHAESS | |
| | | | | TATVRAVFIIDDKGTVRLILYYPMEIGRNIDEILR | |
| | | | | AIRALQLVDKAGVVTPANWPNNELI | |
| | | | | GDKVINPAPRTIKDAKMRLGQPFDWWFTYKE | |
| | | | | VKTT | |
| 2-Cys | Plasmodium | A5K421 | 10- | MPTYVGKEAPFFKAEAVFGDNSFGEVNLTQFI | 24 |
| peroxiredoxin | vivax | | mer | GKKYVLLYFYPLDFTFVCPSEIIALDKA | |
| PV | | | | LDAFHERNVELLGCSVDSKYTHLAWKKTPLAK | |
| | | | | GGIGNIKHTLLSDITKSISKDYNVLFDD | |
| | | | | SVSLRAFVLIDMNGIVQHLLVNNLAIGRSVDEI | |
| | | | | LRIIDAIQHHEKYGDVCPANWQKGKVS | |
| | | | | MKPSEEGVAQYLSTL | |
| Prx LB | Leishmania | A4HCL7 | 10- | MLRRLATKCFQRNVQCRGFAATSPVLNMDY | 25 |
| | braziliensis | | mer | QMYRTATVRDPAPQFSGKAVVDGAIKEINS | |
| | | | | NDYKGKYIVLFFYPMDFTFVCPTEIIAFSDRYLE | |
| | | | | FEKLNTQVIAVSCDSEYSHLAWVNTP | |
| | | | | RKKGGLGEMKIPVLADKSMEIARDYGVLIESA | |
| | | | | GIALRGLFVIDKKGTLRHSTINDLPVGR | |
| | | | | NVDEVLRVVEAFQYADENGDAIPCGWTPGKP | |
| | | | | TLDTKKAGEFFEKNM | |
| Prx AM | Akkermansia | A0A2N8 | 10- | MLLIGKPAPHFSANAVVNGTIVPDFSLDQFKG | 26 |
| | muciniphila | HPY4 | mer | KKYVILFFYPKDFTFVCPTELIGFQEAL | |
| | | | | GEFDKRDVAVVGCSTDSEFSHWAWVNTPRD | |
| | | | | QGGIQGVSYPIVSDINKTISADYGVLAGDE | |
| | | | | EIDEDGNVEVNGELIAYRGLFLIDKDGIVRHQLI | |
| | | | | NDFPLGRSIDEAIRVVDALQHFELYG | |
| | | | | EVCPLGWHKGEAAMTPSHEGVASYLSK | |
| Alkyl | Escherichia | A0A140N | 10- | MSLINTKIKPFKNQAFKNGEFIEITEKDTEGRW | 27 |
| hydroperoxide | coli | C97 | mer | SVFFFYPADFTFVCPTELGDVADHYEE | |
| reductase C EC | | | | LQKLGVDVYAVSTDTHFTHKAWHSSSETIAKI | |
| | | | | KYAMIGDPTGALTRNFDNMREDEGLADR | |
| | | | | ATFVVDPQGIIQAIEVTAEGIGRDASDLLRKIKA | |
| | | | | AQYVASHPGEVCPAKWKEGEATLAPS | |
| | | | | LDLVGKI | |
| Alkyl | Mycobacteriu | P9WQB7 | 12- | MPLLTIGDQFPAYQLTALIGGDLSKVDAKQPG | 28 |
| hydroperoxide | m tuberculosis | | mer | DYFTTITSDEHPGKWRVVFFWPKDFTFV | |
| reductase C MT | H37Rv | | | CPTEIAAFSKLNDEFEDRDAQILGVSIDSEFAHF | |
| | | | | QWRAQHNDLKTLPFPMLSDIKRELSQ | |
| | | | | AAGVLNADGVADRVTFIVDPNNEIQFVSATA | |
| | | | | GSVGRNVDEVLRVLDALQSDELCACNWRK | |
| | | | | GDPTLDAGELLKASA | |

With preference, the monomer of the multimeric protein from the family of the peroxiredoxins is selected from the group consisting of the following: Peroxiredoxin TSA1 from Saccharomyces cerevisiae (SEQ ID NO: 1), Peroxiredoxin from Aeropyrum pernix K1 (SEQ ID NO: 2), Tryparedoxin peroxidase from Crithidia fasciculata (SEQ ID NO: 3), Peroxiredoxin from Chlamydomonas reinhardtii (SEQ ID NO: 4), Chloroplastic 2-Cys peroxiredoxin BAS1, from Arabidopsis thaliana (SEQ ID NO: 5), Mitochondrial peroxiredoxin from Leishmania infantum (SEQ ID NO: 6), Peroxiredoxin-1 from Rattus norvegicus (SEQ ID NO: 7), Tryparedoxin peroxidase from Leishmania major (SEQ ID NO: 8), Peroxiredoxin-4 (isoform 1) from Homo sapiens (SEQ ID NO: 9), Peroxiredoxin TSA2 from Saccharomyces cerevisiae (SEQ ID NO: 10), Alkyl hydroperoxide reductase C from Helicobacter pylori (SEQ ID NO: 11), Peroxiredoxin-2 (isoform 1) from Homo sapiens (SEQ ID NO: 12), Alkyl hydroperoxide reductase C from Salmonella typhimurium (SEQ ID NO: 13), Thioredoxin peroxidase from Schistosoma mansoni (Blood fluke) (SEQ ID NO: 14), Putative tryparedoxin peroxidase from Trypanosoma cruzi (SEQ ID NO: 15), Peroxiredoxin from Pyrococcus furiosus (SEQ ID NO: 16), Peroxiredoxin from Pyrococcus horikoshii (SEQ ID NO: 17), Peroxiredoxin-4 from Mus musculus (SEQ ID NO: 18), Alkyl hydroperoxide reductase C from Amphibacillus xylanus (SEQ ID NO: 19), Peroxiredoxin-1 from Ancylostoma ceylanicum (SEQ ID NO: 20), Alkyl hydroperoxide reductase C from Enterococcus faecalis (SEQ ID NO: 21), Peroxiredoxin 4 from Larimichthys crocea (SEQ ID NO: 22), Peroxiredoxin from Sulfolobus islandicus (SEQ ID NO: 23), Putative 2-Cys peroxiredoxin from Plasmodium vivax (SEQ ID NO: 24), Peroxiredoxin from Leishmania braziliensis (SEQ ID NO: 25), Peroxiredoxin from Akkermansia muciniphila (SEQ ID NO: 26), Alkyl hydroperoxide reductase C from Escherichia coli (SEQ ID NO: 27) and Alkyl hydroperoxide reductase C from Mycobacterium tuberculosis H37Rv (SEQ ID NO: 28) or a functional variant of the latter.

A “peroxiredoxin monomer” or “peroxiredoxin” is any monomer of natural peroxiredoxins, but also the functional variants thereof which retain in particular the capacity to undergo multimerization to form a decameric or dodecameric ring, the N- and C-terminal ends of the monomers being exposed on the outside of this ring.

Preferably, as used herein, the term “variant” or “functional variant” denotes a polypeptide having an amino acid sequence which exhibits at least 70, 75, 80, 85, 90, 95 or 99% sequence identity with the sequence of amino acids in the peroxiredoxin as described above, more particularly with one of the sequences selected from the group consisting of SEQ ID NO: 1-28 and which retains its capacity to undergo multimerization to form a decameric or dodecameric ring, the N- and C-terminal ends of the monomers being exposed on the outside of this ring.

As used herein, the term “sequence identity” or “identity” refers to the number (%) of correspondences (identical amino acid residues) in positions originating from an alignment of two polypeptide sequences. Sequence identity is determined by comparing the sequences when they are aligned so as to maximize the overlap and the identity while minimizing the gaps between the sequences. More particularly, the sequence identity may be determined using a certain number of global or local mathematical alignment algorithms, depending on the length of the two sequences. Sequences of similar lengths are preferably aligned using a global alignment algorithm (for example, the algorithm of Needleman and Wunsch; Needleman and Wunsch, 1970) which aligns the sequences optimally over the whole length, whereas sequences having substantially different lengths are preferably aligned using a local alignment algorithm (for example, the algorithm of Smith and Waterman (Smith and Waterman, 1981) or the algorithm of Altschul (Altschul et al, 1997; Altschul et al, 2005)). Alignment for the purposes of determining the percentage identity of the amino acid sequences may be performed in different ways which fall within the competence of the skilled person, as for example by using digital software which is accessible by the public on websites such as http://blast.ncbi.nlm.nih.gov/ or http://www.ebi.ac.uk/Tools/emboss/. Specialists in this field are able to determine the appropriate parameters for measuring the alignment, including the algorithms needed in order to obtain maximum alignment over the whole length of the sequences compared. For the purposes of the present document, the values for amino acid sequence identity in percent refer to the values generated using the EMBOSS Needle pairwise sequence alignment program, which creates an optimal global alignment of two sequences using the Needleman-Wunsch algorithm, in which all of the search parameters are regulated to values by default, i.e. Scoring matrix=BLOSUM62, Open space=10, Extended space=0.5, End space penalty=false, End open space=10, and End extended space=0.5.

The term “variant” or “functional variant” preferably denotes a polypeptide having an amino acid sequence which differs from a sequence of the sequences SEQ ID NO: 1 to 28 in one or more conservative substitutions.

By “substituted” or “modified”, the present invention understands amino acids which have been altered or modified on the basis of natural amino acids.

The term “conservative substitution” as used here denotes the replacement of one amino acid residue with another, without adversely affecting the conformation and the overall function of the protein, including, but without limitation to, the replacement of one amino acid with another having similar properties (such as, for example, polarity, hydrogen bonding potential, acidity, base acidity, shape, hydrophobicity, aromaticity, etc.).

Examples of conservative substitutions are found in the groups of basic amino acids (arginine, lysine and histidine), of acidic amino acids (glutamic acid and aspartic acid), of polar amino acids (glutamine and asparagine), of hydrophobic amino acids (methionine, leucine, isoleucine and valine), of aromatic amino acids (phenylalanine, tryptophan and tyrosine) and of small amino acids (glycine, alanine, serine and threonine).

The peroxiredoxin or the functional variant of peroxiredoxin in the present patent application is a peroxiredoxin which does not exhibit redox activity. More particularly, the peroxiredoxins or peroxiredoxin variants comprise a mutated peroxidatic cysteine and/or “resolving” residue. The peroxidatic cysteine and the “resolving” cysteine of peroxiredoxin have been characterized for a great number of peroxiredoxin proteins and are well known to the skilled person (Nelson et al. Proteins, 2012, 79(3):947-964; http://csb.wfu.edu/prex/search.php). More particularly, the C_{R} is conserved in the same position as C172 in human PrxII. As an example, the peroxidatic and resolving cysteines for a large number of peroxiredoxin proteins are indicated in table 1.

The peroxiredoxin monomer as described above preferably exhibits a peroxidatic and/or “resolving” cysteine which have been mutated by any other amino acids, preferably by an alanine or a serine.

The present patent application preferably relates to the peroxiredoxin monomer TSA1 from Saccharomyces cerevisiae, preferably which exhibits a cysteine mutated in position 48 and/or 171 of the sequence SEQ ID NO: 1 by any other amino acids, preferably by an alanine or a serine, preferably the mutated Prx monomer comprising or consisting of sequence SEQ ID NO: 29.

The inventors have demonstrated that the peroxiredoxin from Pyrococcus furiosus, which is particularly stable and conserves its decameric structure even at low concentration, is particularly advantageous as a scaffold protein. Therefore, in another preferred embodiment, the present patent application relates to the peroxiredoxin monomer from Pyrococcus furiosus, preferably which exhibits a cysteine mutated in position 46 and/or 211 of the sequence SEQ ID NO: 16 by any other amino acids, preferably by an alanine or a serine, preferably the mutated Prx monomer comprising or consisting of sequence SEQ ID NO: 52.

Peroxiredoxins capable of undergoing multimerization to form a decameric or dodecameric ring exhibit floating ends in N- and C-terminal positions of each of the monomers. The one or more proteins of interest may therefore be linked to one or to the two N- and C-terminal end(s) of one or more monomers of the peroxiredoxin, as described above.

The multimeric protein from the family of the peroxiredoxins according to the invention preferably comprises at least two different proteins of interest which are linked to one or two N- and C-terminal end(s) of one or more peroxiredoxin monomers having no redox activity.

A protein of interest comprehends any proteins whose activity or function is improved when they are assembled into a protein complex. The proteins of interest preferably have a size of less than 50 kDa, preferably less than 40, 30, 20 or 10 kDa, preferably 20 kDa.

The proteins of interest according to the invention are preferably enzymes or proteins of a metabolic pathway.

In one particular embodiment, the proteins of interest are proteins from the same metabolic pathway and the scaffold protein is used for augmenting the efficacy of synthesis. For example, the proteins of the same metabolic pathway are proteins which allow the synthesis of molecules of interest such as, for example: catechin, D-glucaric acid, H2, hydroquinone, resveratrol, butyrate, γ-aminobutyric acid and mevalonate, (2S,5S)-hexanediol, mono(2-hydroxyethyl) terephthalate (MHET), terephthalic acid and ethylene glycol.

In another particular embodiment, the proteins of interest are enzymes and the scaffold protein is used for augmenting the catalytic activity of the enzymes. The enzymes are preferably enzymes having a polymeric substrate. For example, the enzymes may be selected from the group consisting of the following: PET hydrolases (PETase), lysozyme, MHETase, alcohol dehydrogenase, lytic polysaccharide monooxygenases (LPMO) and endopeptidases.

In one particular case, the proteins of interest are not reporter proteins.

The term “reporter protein” denotes a protein which possesses a feature allowing it to be observed or quantified, by detection of bioluminescence (for example, fluorescence), of enzymatic activity or by recognition by an antibody, for example. A reporter protein may therefore be selected from fluorescent proteins, enzymes having an action giving rise to the appearance of a colored product or of a readily characterizable phenotype, or any other protein having any other quantifiable activity, or short sequences of amino acids referred to as tags. The reporter proteins are selected from luciferase, beta-galactosidase (LacZ) or a hydrolase (β-glucuronidase, alkaline phosphatase), chloramphenicol acetyltransferase (CAT) and beta-lactamase (TEM-1), the tag HA (human influenza hemagglutinin), FLAG, polyHis6 and fluorescent proteins.

The proteins of interest may be linked to the N- or C-terminal ends of the monomer of peroxiredoxin by covalent or noncovalent linkages.

In one particular embodiment, the protein of interest is fused covalently to the N- or C-terminal end of the monomer of peroxiredoxin.

The term “fusion protein” used in the present patent application refers to a protein which comprises at least two different polypeptides not originating from the same protein.

The two proteins may be fused directly or via a peptide sequence, called a linker, which enables the various proteins or protein fragments to be linked such that the protein adopts a better conformation for the activity of the proteins. More particularly, the nucleotide sequence encodes a peptide sequence of 1, 2, 3, 4, 5, 10, 15, 20, 30, 40 or 50 amino acids. In one preferred embodiment, the linker encodes a peptide sequence consisting of GGGS, GGGSGGGS (SEQ ID NO: 30) or GGGSGGGSGGGS (SEQ ID NO: 31).

In another particular embodiment, the protein of interest is linked noncovalently to the N- or C-terminal end of the monomer of peroxiredoxin, preferably via an adapter/peptide ligand pair. The adapter/peptide ligand pair consists of an adapter region of a protein which has a high affinity for a peptide ligand that exhibits a specific sequence motif enabling effective coupling of the two proteins to which they are respectively linked.

The use of an adapter/peptide ligand pair makes it possible to control the number and type of proteins of interest that are linked to the Prx monomers of the scaffold protein and to prevent any steric hindrance of the proteins of interest on the scaffold protein.

Adapter/peptide ligand pairs which can be used in the context of the invention are well known to the skilled person and include, for example, the domains SH3, SH2, PDZ, GBD (GTPase-binding domain) (Horn A. H. C. et al. 2015, Front Bioeng Biotechnol. 3:191).

The inventors have also characterized new adapter/peptide ligand pairs comprising the linking domains of the proteins Rsa1 (Uniprot ID: Q08932), NUFIP1 (Uniprot ID: Q9UHK0), HSP90 (Uniprot IDs: P08238, P07900), HSP70 (Uniprot ID: PODMV8), RPAP3 (Uniprot: Q9H6T3), SPAG1 (Uniprot ID: Q07617) or Tah1 (Uniprot ID: P25638).

In one preferred embodiment, the adapter/peptide ligand pair is selected from the following pairs (cf. Table 2):
HSP90C of SEQ ID NO: 32 or 33 and SPAG1-622742 of SEQ ID NO: 34;
HSP90C of SEQ ID NO: 32 or 33 and SPAG1-206327opt of SEQ ID NO: 35;
HSP90C of SEQ ID NO: 32 or 33 and RPAP3-281396 of SEQ ID NO: 36;
HSP90C of SEQ ID 32 or 33 and Tah1 of SEQ ID NO: 37;
HSP70C of SEQ ID NO: 38 and SPAG1-622742 of SEQ ID NO: 34;
HSP70C of SEQ ID NO: 38 and SPAG1-206327opt of SEQ ID NO: 35;
ySNU13 of SEQ ID NO: 39 and Rsa1-238259 of SEQ ID NO: 40;
hSNU13 of SEQ ID NO: 41 and NUFIP1-233255 of SEQ ID NO: 42;
Tah1-93111 of SEQ ID NO: 43 and Pih1-258344 of SEQ ID NO: 44;
RPAP3-396455 of SEQ ID NO: 45 and PIH1D1-199290 of SEQ ID NO: 46;
ZNHIT3-85155 of SEQ ID NO: 47 and NUFIP1-462495 of SEQ ID NO: 48;
Hit1-70164 of SEQ ID NO: 49 and Rsa1-317352 of SEQ ID NO: 50.

| Name | SEQ ID | Peptide sequence |
|---|---|---|
| HSP90C | 32 | DASRMEEVD |
| HSP90C | 33 | DTSRMEEVD |
| SPAG1-622742 | 34 | TFKALKEEGNQCVNDKNYKDALSKYSECLKINNKECAIYTNRALCYLKL |
| | | CQFEEAKQDCDQALQLADGNVKAFYRRALAHKGLKNYQKSLIDLNKV |
| | | ILLDPSIIEAKMELEEVTRLLNLKD |
| SPAG1-206327opt | 35 | DYLATREKEKGNEAFNSGDYEEAVMYYTRSISALPTVVAYNNRAQAYI |
| | | KLQNWNSAEQDCEKVLELEPGNVKALLRRATAYKHQNKLREAREDLK |
| | | KVLKVEPDNDLAKKTLSEVERDLKNSE |
| RPAP3-281396 | 36 | QAISEKDRGNGFFKEGKYERAIECYTRGIAADGANALLPANRAMAYLK |
| | | IQKYEEAEKDCTQAILLDGSYSKAFARRGTARTFLGKLNEAKQDFETVL |
| | | LLEPGNKQAVTELSKIKKE |
| Tah1 | 37 | MSQFEKQKEQGNSLFKQGLYREAVHCYDQLITAQPQNPVGYSNKAM |
| | | ALIKLGEYTQAIQMCQQGLRYTSTAEHVAIRSKLQYRLELAQGAVGSV |
| | | QIPVVEVDELPEGYDRS |
| HSP70C | 38 | SGPTIEEVD |
| ySNU13 | 39 | MSAPNPKAFPLADAALTQQILDVVQQAANLRQLKKGANEATKTLNR |
| | | GISEFIIMAADCEPIEILLHLPLLCEDKNVPYVFVPSRVALGRACGVSRPV |
| | | IAASITTNDASAIKTQIYAVKDKIETLLI |
| Rsa1-238259 | 40 | TDEDVKKWREERKKMWLLKISN |
| hSNU13 | 41 | MTEADVNPKAYPLADAHLTKKLLDLVQQSCNYKQLRKGANEATKTLN |
| | | RGISEFIVMAADAEPLEIILHLPLLCEDKNVPYVFVRSKQALGRACGVSR |
| | | PVIACSVTIKEGSQLKQQIQSIQQSIERLLV |
| NUFIP1-233255 | 42 | TPEEIARWREERRKNYPTLANIER |
| Tah1-93111 | 43 | SVQIPVVEVDELPEGYDRS |
| Pih1-258344 | 44 | HEQQEDVPEYEVKMKRFKGAAYKLRILIENKAPNSKPDRFSPSYNFAE |
| | | NILYINGKLSIPLPRDIVVNAADIKIFHIRKERTLYIYI |
| RPAP3-396455 | 45 | ELIEKGHWDDVFLDSTQRQNVVKPIDNPPHPGSTKPLKKVIIEETGNLI |
| | | QTIDVPDSTTA |
| PIH1D1-199290 | 46 | GRAESGPEKPHLNLWLEAPDLLLAEIDLPKLDGALGLSLEIGENRLVMG |
| | | GPQQLYHLDAYIPLQINSHESKAAFHRKRKQLMVAMPLLLVPS |
| ZNHIT3-85155 | 47 | DRVSLQNLKNLGESATLRSLLLNPHLRQLMVNLDQGEDKAKLMRAY |
| | | MQEPLFVEFADCCLGIVEPSQNEES |
| NUFIP1-462495 | 48 | DIRHERNVILQCVRYIIKKDFFGLDTNSAKSKDV |
| Hit1-70164 | 49 | MNKTLKTKAFDDIYQNSAELQELLKYNTVKFHLAKVYRILSSTVNDGSS |
| | | GKMNSDLQKELAVNYLNTLRYGGIHYNEAIEEFCQILLDKLNAVKK |
| Rsa1-317352 | 50 | FANENSQLLDFIRELGDVGLLEYELSQQEKDVLFGS |

Table 2: Sequences used for the adapter/peptide ligand pairs.

More particularly, the adapter domain and/or the peptide ligand as described above is fused to the monomer of peroxiredoxin, preferably to one or to the two N- or C-terminal end(s), and the adapter domain and/or the peptide ligand that corresponds is fused to the protein of interest such that the monomer of peroxiredoxin and the protein of interest are linked to one another via the linkage of the adapter/peptide ligand pair.

In one privileged embodiment, the protein is synthesized using recombinant techniques. In this case, a nucleic acid construction comprising or consisting of a nucleic acid sequence encoding the proteins as described above is used and expressed in host cells. The nucleic acids and expression vectors which may be used for producing the proteins according to the present patent application are described below.

### Nucleic Acids and Expression Vector

The present patent application further relates to one or more nucleotide constructions encoding for a multimeric protein as described above.

The terms “nucleic acid” or “nucleotide sequence” may be used interchangeably to refer to any molecule composed of or comprising nucleic acids. A nucleic acid may be an oligonucleotide or a polynucleotide. A nucleotide sequence may be a DNA or a RNA. A nucleotide sequence may be modified chemically or artificially. The nucleotide sequences may comprise nucleic acids, peptides, morpholinos, blocked nucleic acids, and also glycol nucleic acids and threose nucleic acids. Each of these sequences differs from the natural DNA or RNA in the nature of the skeleton. Phosphorothioate nucleotides may be used. Other nucleic acid analogs such as methylphosphonates, phosphoramidates, phosphorodithioates, N3′P5′-phosphoramidates and phosphorothioate oligoribonucleotides and the analogs 2′-O-allyl- and 2′-O-methylribonucleotide methylphosphonates may be used in the context of the invention.

In one particular embodiment, the present patent application relates to a nucleotide construction encoding for a peroxiredoxin monomer fused to one or two N- and C-terminal end(s) with a protein of interest, preferably via a linker.

The nucleotide sequence encoding the peroxiredoxin monomer as described above is preferably fused to the nucleotide sequence encoding the protein of interest via a “linker”, preferably a nucleotide sequence which encodes a peptide sequence that allows the various proteins or protein fragments to be linked such that the protein adopts a better conformation for the activity of the proteins. More particularly, the nucleotide sequence encodes a peptide sequence of 1, 2, 3, 4, 5, 10, 15, 20, 30, 40 or 50 amino acids. In one preferred embodiment, the linker encodes a peptide sequence GGSGGS (SEQ ID NO: 30), GGGSGGGSGGS (SEQ ID NO: 31) or GGGS.

The fusion of two or more nucleotide sequences refers to the combination of two or more nucleotide sequences encoding different proteins or protein fragments, with the translation of the two fused genes giving a single functional polypeptide. In the context of the invention, a peroxiredoxin monomer is fused to a protein of interest to form a multimeric “scaffold” protein which allows the protein or proteins of interest to be complexed.

In another particular embodiment, the present patent application relates to a nucleotide construction encoding for a peroxiredoxin monomer fused at one or at the two N- and C-terminal end(s) with a first peptide sequence of an adapter domain as defined above, and a nucleotide construction encoding for a protein of interest fused to a corresponding peptide ligand of the adapter/peptide ligand pair. The peroxiredoxin monomer is then capable of linking noncovalently via the adapter/peptide ligand pair to the one or more proteins of interest and of forming a multimeric “scaffold” protein which allows the one or more proteins of interest to be complexed.

The nucleotide sequence as described refers preferably to the complementary DNA, also called cDNA, which encodes the monomer of the peroxiredoxin or the protein of interest. The nucleotide construction of the present patent application preferably comprises a sequence encoding a peroxiredoxin monomer as described above preferably comprising a sequence SEQ ID NO: 51 encoding a peroxiredoxin monomer TSA1 from S. cerevisiae mutated in position 48 and 171.

In another preferred embodiment, the nucleotide construction of the present patent application comprises a sequence SEQ ID NO: 53 encoding a peroxiredoxin monomer from Pyrococcus furiosus mutated in position 46 and 211.

The present patent application further relates to a vector comprising the nucleotide construction as described above.

A “vector” here refers to a DNA molecule which may equally have a single-strand or double-strand form.

A recombinant vector according to the invention is preferably a plasmid vector or an integration vector. In certain embodiments of the invention, the vector is a plasmid. A “plasmid” here refers to a double-stranded circular DNA molecule which possesses a replication origin so that it can replicate autonomously in the cell, and a selection gene so that it is not lost by the organism in the course of cell multiplications. A large number of vectors are known per se; the selection of an appropriate vector depends on the intended use of this vector (for example, replication of the sequence of interest, expression of this sequence, maintenance of this sequence in extrachromosomal form, or else integration into the chromosomal apparatus of the host), and also on the nature of the host cell.

Said vector is preferably an expression vector comprising all of the elements needed for the expression of the genes of interest as defined above. For example, said vector comprises an expression cassette including at least one gene of interest as defined above, under the control of appropriate sequences regulating the transcription and possibly the translation (promoter, activator, intron, start codon (ATG), codon stop, polyadenylation signal, splicing site).

The vector is preferably a DNA plasmid vector. In one particular embodiment, the vectors are adapted for use in prokaryotic host cells and are preferably selected from the following: ACYC184, pBeloBacll, pBR332, pBAD33, pBBR1MCS and its derivatives, pSC101, SuperCos (cosmid), pWE15 (cosmid), pTrc99A, pBAD24, vectors containing a ColE1 replication origin and its derivatives, pUC, pBluescript, pCARGHO, pET, pGEM, pnEA, pnYK, pnCS and pTZ vectors.

Further to the coding region, the gene of interest may comprise or be combined with one or more elements which facilitate or augment the expression of said gene, such as activating sequences, response elements, insulators, polyadenylation signals and/or any other functional element.

The nucleotide sequences encoding the peroxiredoxin monomer and/or the proteins of interest may be inserted into an expression vector, under the transcription control of a promoter for permitting the expression of the proteins.

A “promoter” refers to any polynucleotide which is capable of positively regulating the expression, in a cell, of a nucleotide sequence to which it is linked operationally. A promoter or a promoter sequence is a DNA region which is situated close to a gene and is indispensable to the transcription of the DNA into RNA. Promoter sequences are generally situated upstream of the transcription initiation site. Promoter sequences correspond to the region of initial attachment of the RNA polymerase before it initiates the synthesis of RNA.

### Host Cells

The present patent application further relates to a host cell comprising the multimeric protein, the nucleotide construction or the vector, all as described above.

The host cells may be eukaryotic or prokaryotic cells.

Eukaryotic cells comprise, in particular, animal cells, fungal cells, insect cells, plant cells and algal cells.

Eukaryotic host cells are preferably selected from the group consisting of the following: Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorphs, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, Spodoptera frugiperda Sf9, Spodoptera frugiperda Sf21 and Chlamydomonas reinhardtii.

Prokaryotic cells are preferably selected from the group consisting of Escherichia coli, Lactobacillus sp., Salmonella sp., Shigella sp., Rhodococcus sp., Bacillus sp. and Pseudomonas sp.

### Use of the Multimeric Protein

The multimeric protein from the family of the peroxiredoxins as described above is used as a scaffold protein. The scaffold protein will enable one or more proteins of interest to be complexed in order to improve the function of the one or more proteins of interest.

The scaffold protein preferably enables the synthesis of a molecule of interest to be augmented, by complexing one or more metabolic pathway proteins on the scaffold protein. Improving the function, when the protein of interest is a metabolic pathway protein, refers to augmenting the synthesis of the molecule of interest, which corresponds to the end product or an intermediate product of the metabolic pathway. More particularly, the use of the scaffold protein according to the invention permits an augmentation of at least 2, 3, 4, 5, 6, 7, 8, 9, 10, 15, 20, 25, 30, 40 or 50 times the level of metabolic production of the molecule of interest by comparison with production in the presence of free enzymes.

The molecules of interest include, without limitation: catechin, D-glucaric acid, H2, hydroquinone, resveratrol, butyrate, γ-aminobutyric acid, mevalonate, (2S,5S) hexanediol, mono(2-hydroxyethyl) terephthalate (MHET), terephthalic acid and ethylene glycol.

When the protein of interest is an enzyme, improving the function refers to augmenting the catalytic activity of the enzyme. The scaffold protein therefore enables the concentration and the stability of the enzyme to be augmented, and therefore augments its catalytic activity.

In one particular embodiment, the protein of interest is a hydrolytic enzyme, preferably a hydrolytic enzyme with a polymeric substrate. The hydrolytic enzyme is preferably an enzyme selected from the following: PET hydrolase, MHETase, lysozyme, alcohol dehydrogenase, LPMO and endopeptidase.

A better appreciation of the invention will be had with the aid of the examples which follow, these examples not wishing to impose any limitation, and merely documenting certain embodiments and certain advantageous properties of the invention.

### Examples

### 1. Example of Covalent Grafting

In the example below, the CRD_{SAT} domain of human galectin-3 was grafted covalently onto variant C171A of the TSA1 protein from S. cerevisiae. For this, plasmids suitable for the overexpression of protein by Escherichia coli were synthesized. The oligonucleotide sequence of the CRD_{SAT} domain (Kriznik et al. 2019, Biotechnol. J., doi: 10.1002/biot.201800214) was placed on these plasmids, either upstream (Nter position) or downstream (Cter position), or on either side of the oligonucleotide sequence of TSA1-C171A. When placed in the Cter position, the sequence of the CRD_{SAT} is preceded by a non-native linker region of protein sequence GGGSGGGSGGGS (i.e. (GGGS)3, SEQ ID NO: 31).

a. Protocol

Each plasmid was introduced by heat shock for 60 seconds at 42° C. into a competent E. coli BL21(DE3) pRARE2 Ca²⁺ strain. The transformed clones were selected on solid LB-agar medium supplemented with ampicillin (A) and chloramphenicol (C). After one night at 37° C., an isolated colony is cultured for 16 hours at 37° C. with stirring in 50 mL of LB-AC medium. 10 mL of this culture are introduced into 500 mL of LB-AC medium and then cultured at 37° C. with stirring until the absorbance of the solution, measured at a wavelength of 600 nm, attains a value of 0.6. The overexpression of the recombinant proteins encoded by the plasmids is induced by addition of isopropyl β-D-1-thiogalactopyranoside (IPTG, final concentration of 0.25 mM). The culture is then placed at 20° C. with stirring for 16 hours and then centrifuged for 45 minutes at 4000 g. The bacterial pellet is taken up in 20 mL of buffer 1 (25 mM HEPES, pH 7.5, 300 mM NaCl, 0.5 mM TCEP) and lyzed by sonication. The sonicate is centrifuged for 30 minutes at 4° C. and at 20 000 g and then the supernatant is incubated for 30 minutes at 4° C. with 1 mL of 50% lactose-sepharose resin, equilibrated with buffer 1 beforehand. The resin is washed three times with buffer 1. The recombinant proteins are eluted by addition of three times 1.5 mL of buffer 1 containing D-lactose at a concentration of 200 mM. Samples are taken at each purification step (dissolving, fixing, eluting) and are analyzed by SDS-PAGE. If necessary, the fractions containing the protein of interest are assembled and concentrated until the volume is 0.25 mL. This solution is injected into a Superose 6 Increase analytical column (S6I, GE Healthcare) equilibrated with buffer 2 (20 mM HEPES, pH 7.5, 100 mM NaCl) beforehand. The proteins are eluted according to their size with buffer 2. Their elution is monitored by measuring the absorbance at 280 nm. Fractions of 0.5 mL are collected at the column exit. The presence of the proteins of interest in the fractions is monitored by SDS-PAGE.

b. Results

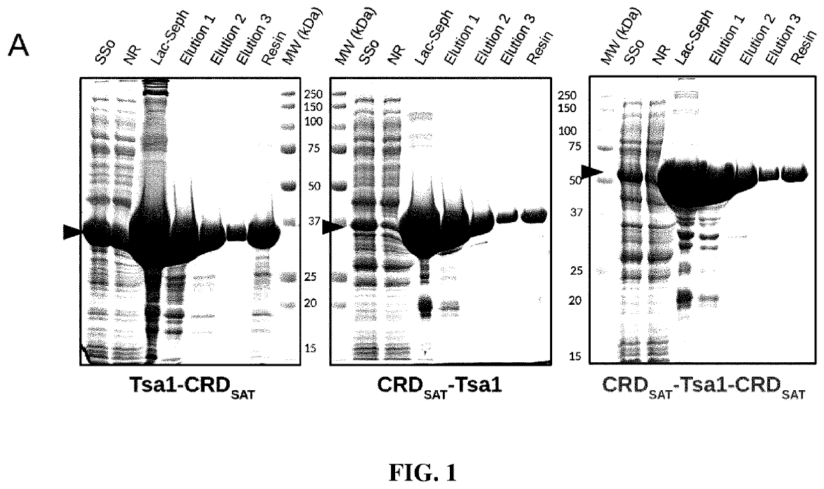

The presence of the proteins CRD_{SAT}-TSA1, TSA1-CRD_{SAT}, CRD_{SAT}-TSA1-CRD_{SAT} in the sonication supernatant, on the Lactose-Sepharose resin and in the elution fractions from the Lactose-Sepharose resin was detected (FIG. 1A). SDS-PAGE analysis of the majority peaks of each elution shows that the fractions of these peaks contain the proteins CRD_{SAT}-TSA1, TSA1-CRD_{SAT} or CRD_{SAT}-TSA1-CRD_{SAT} with a high degree of purity. The retention volumes on the S6I column and the migration profile of the proteins on SDS-PAGE are in agreement with the molecular weight of the proteins CRD_{SAT}-TSA1, TSA1-CRD_{SAT} or CRD_{SAT}-TSA1-CRD_{SAT} (FIGS. 1B and 1C). Dynamic light scattering (DLS) analysis of the S6I fractions containing the proteins of interest was carried out at 20° C. by a NanoSizer instrument (Malvern). For each protein of interest, this analysis shows a monodisperse peak which characterizes a species very much in the majority in solution. As demonstrated by the measurement carried out with the decameric wild-type protein TSA1, the size of the proteins CRD_{SAT}-TSA1, TSA1-CRD_{SAT} and CRD_{SAT}-TSA1-CRD_{SAT} is compatible with a TSA1 decamer adorned at Nter, Cter or Nter/Cter by the CRD_{SAT} domain (FIG. 1D).

### 2. Example of Noncovalent Grafting

a. Protocol

For the production of the recombinant proteins, a pCARGHO2-type plasmid was designed which encodes the oligonucleotide sequence of the protein TSA1-C171A, followed by a GGGSGGGS linker (SEQ ID NO: 30) or GGGSGGGSGGGS linker (SEQ ID NO: 31) and then by the peptide sequence DASRMEEVD (SEQ ID NO: 32) obtained from the human protein HSP90 (HSP90pep). A pnEA vector enables encoding of the TPR3 domain of the human protein SPAG1 (corresponding to the 622-742 region of the protein, SEQ ID NO: 34) fused to a 6-histidines (6×HIS) label in N-terminal position. As detailed above, 30 mL cultures are produced of E. coli BL21(DE3) pRARE2 strains which in an LB medium have overexpressed the recombinant proteins CRD_{SAT}-TSA1-(GGGS)_{2 or 3}-HSP90pep and 6×HIS-SPAG1-TPR3.

30 mL of bacterial culture having expressed 6×HIS-SPAG1-TPR3 are pelletized, taken up in 1.5 mL of buffer 3 (25 mM HEPES, pH 7.5, 300 mM NaCl, 10 mM imidazole, 0.5 mM TCEP) and centrifuged for 20 minutes at 16 000 g. 500 μL of supernatant are brought into contact with 150 μL of a TALON resin suspension for 20 minutes at 4° C. The resin is washed with three times 500 μL of buffer 1. 30 mL of bacterial culture having CRD_{SAT}-TSA1-(GGGS)2 or 3-HSP90pep are taken up in 1.5 mL of buffer 1 and centrifuged for 20 minutes at 16 000 g. 500 μL of supernatant are brought into contact for 30 minutes at 4° C. with the TALON beads which have fixed 6×HIS-SPAG1-TPR3. The resin is washed with three times 500 μL of buffer 3. The proteins fixed to the beads are eluted with 150 μL of buffer 3 admixed with 300 mM Imidazole. Controls which involve directly contacting 150 μL of TALON beads and 500 μL of supernatant containing CRD_{SAT}-TSA1-(GGGS)2 or 3-HSP90pep (and treated under conditions strictly identical to those used above) make it possible to evaluate the degree of fixing which is nonspecific to the resin of the proteins without 6×HIS labelling.

b. Results

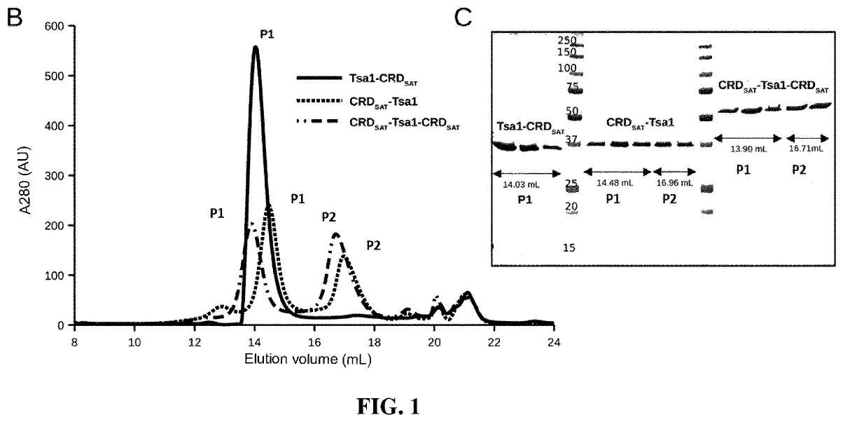

The reconstitution of a complex of CRD_{SAT}-TSA1-(GGGS)2 or 3-HSP90pep with 6×HIS-SPAG1-TPR3 is monitored by SDS-PAGE (FIG. 2). The inventors have observed that the proteins of interest are present in the sonication supernatants and that the fixing of 6×HIS-SPAG1-TPR3 to the TALON beads is specific. By comparison with the control experiments, the inventors have shown that the proteins CRD_{SAT}-TSA1-(GGGS)2 or 3-HSP90pep are more readily retained on the TALON beads which have fixed 6×HIS-SPAG1-TPR3 than on the bare TALON beads. The proteins CRD_{SAT}-TSA1-(GGGS)2 or 3-HSP90pep and 6×HIS-SPAG1-TPR3 are coeluted stoichiometrically by the addition of a high concentration of Imidazole. The small amount of proteins eluted in the control experiments suggests that a specific complex of CRD_{SAT}-TSA1-(GGGS)_{2 or 3}-HSP90pep with 6×HIS-SPAG1-TPR3 is formed in solution.

A noncovalent complex is reconstituted by coexpressing the CRD_{SAT}-TSA1-(GGGS)_{2 or 3}-HSP90pep and 6×HIS-SPAG1-TPR3 in an E. coli BL21(DE3) bacterial strain. For this, the plasmids which encode the proteins should possess compatible replication origins. Each plasmid provides a single resistance to an antibiotic so as to select the bacteria which have been cotransformed by the two plasmids. The noncovalent complex is purified by one or other of the labels, preferably via the 6×HIS label in the case cited above.

### 3. Example of Grafting of Enzyme of Interest

a. Equipment

### Production and Purification of Recombinant Proteins

The peroxiredoxin-type scaffolding and also the enzymes of interest for grafting are produced heterologously in Escherichia coli BL21 (DE3) pRARE2 by virtue of inducible expression plasmids. The proteins are purified in a 25 mM HEPES (pH 7.5), 150 mM NaCl buffer, starting from the soluble fraction of the cellular extract, using two successive chromatographies. A first metal affinity chromatography (GE Healthcare HiTrap Talon Crude 5 mL) is followed by a size exclusion chromatography (Superose® 6 Increase 10/300 GL).

### Assembly of the Enzymatic Scaffolding

The scaffolding selected corresponds to the Prx1 from Pyrococcus furiosus (Pfu-Prx) in which the cysteine residues have been mutated into serine residues (SEQ ID NO: 52). The enzymes of interest selected are the protease PreScission 3C and a PET hydrolase (or PETase) obtained from compost (SEQ ID NO: 54 and 55). The assembly of the enzyme on the scaffolding is produced either by gene fusion or by using a protein/adapter peptide pair as described above. A polyglycine-type linker separates the scaffolding from the enzyme of interest.

b. Results

### Analysis of Protein Particle Size by Dynamic Light Scattering (DLS).

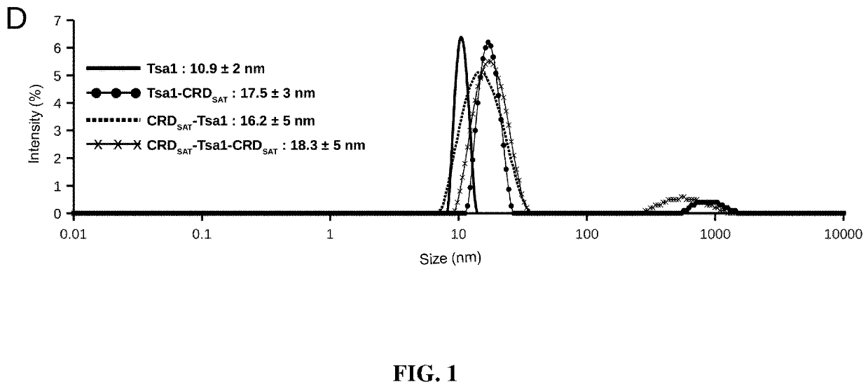

The DLS experiments were carried out at 20° C. in a 25 mM HEPES (pH 7.5), 150 mM NaCl buffer, using a Zetasizer Nano ZS instrument (Malvern Panalytical) and a small-volume quartz cell. The results are presented in FIG. 3.

The size of the particles, measured at 14.2 nm for Pfu-Prx, demonstrates the decameric ring structuring of this protein. Accordingly, the grafting of the 3C protease and the PETase onto Pfu-Prx brings about the formation of larger-size particles, of 27.8 and 20.3 nm respectively. This analysis shows that the scaffolding of the enzymes of interest on a decameric Pfu-Prx ring has been carried out correctly.

### Monitoring of Dissociation by Isothermal Titration Microcalorimetry (ITC)

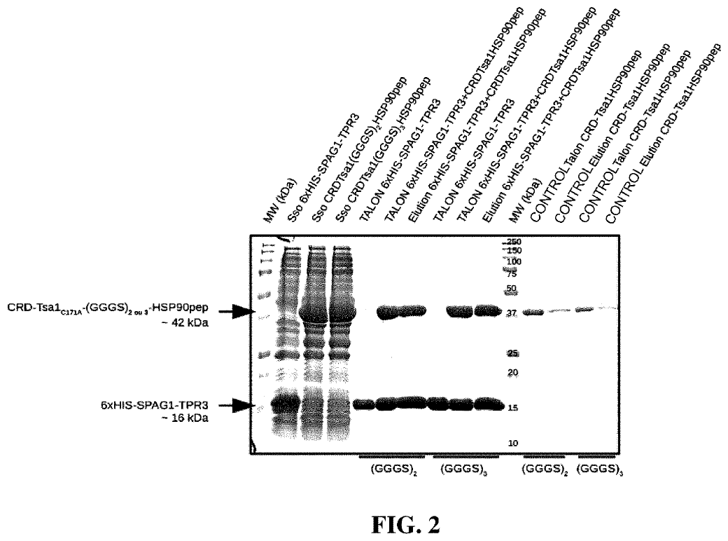

The peroxiredoxin Tsa1 from S. cerevisiae and Pfu-Prx were injected into the cell of a microcalorimeter, using a 100 μM VP-ITC stock solution (Malvern). The experiments are carried out at 25° C. in a 25 mM HEPES (pH 7.5), 150 mM NaCl buffer. The results are represented in FIG. 4.

The heat release measurements enable a critical transition concentration (or CTC) to be determined. This concentration corresponds to the dissociation of 50% of the decamers in solution. A value of 2.8 μM was determined for Tsa1 (gray). In contrast, no value could be determined for Pfu-Prx (black).

This result confirms that Pfu-Prx conserves its decameric ring structuring, even at very low concentration (below the μ-molar threshold). This parameter is advantageous for the scaffolding function. Pfu-Prx is therefore a tool which is very suitable for the invention.

### Monitoring of Thermal Denaturation by Differential Scanning Microcalorimetry (DSC).

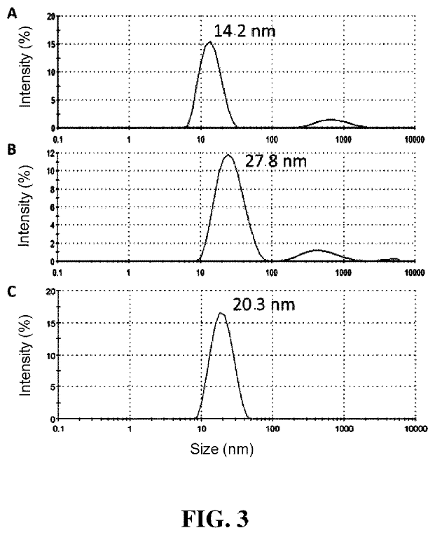

Pfu-Prx was injected into the cell of a differential scanning microcalorimeter (DSC), using a stock solution with a 0.5 mg/ml concentration and at a pressure of 2 atm. The experiment is carried out in a 25 mM HEPES (pH 7.5), 150 mM NaCl buffer. The results are presented in FIG. 5.

The thermogram recorded shows two specific semi-denaturation temperatures (or Tm), which may be interpreted as i) the temperature at which the decamer undergoes dissociation to a dimer (93.4° C.), and ii) the temperature at which the monomers are denatured (107.8° C.). These very high temperatures show the very great stability of the decameric edifice of Pfu-Prx.

Stability at high temperature is an advantage for industrial application. Moreover, this structural stability suggests the long-term integrity of the scaffolding.

### Proteolytic Activity of the Scaffolded PreScission 3C Protease

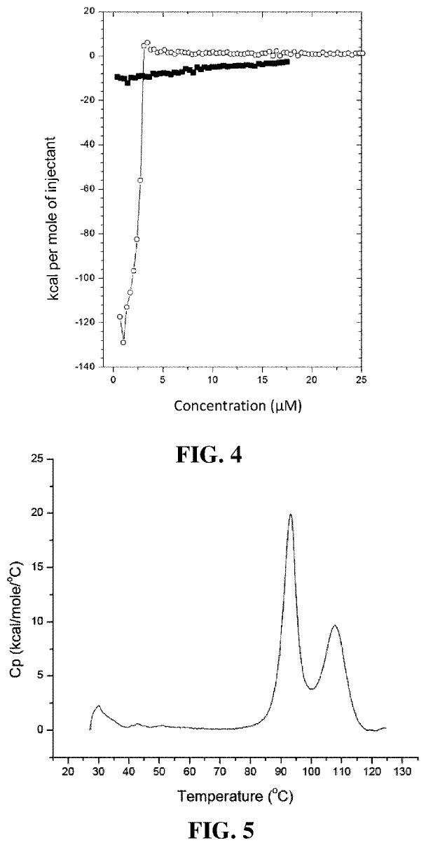

The proteolytic activity of PreScission 3C scaffolded on Pfu-Prx was evaluated using a 6His-SPAG1-TPR3 substrate, the latter possessing a specific cleavage site for said protease. The substrate, at a concentration of 1 mg/mL, and the scaffolded protease, at a concentration of 2 μM, are brought into contact in 1 mL of 25 mM HEPES (pH 7.5), 150 mM NaCl buffer for 60 minutes at 10° C. Analysis is carried out by electrophoresis on denaturing gel and staining with Coomassie blue. The results are presented in FIG. 6.

The increasing appearance over time of a specific cleavage product shows that the protease scaffolded on Pfu-Prx exhibits proteolytic activity. The absence of spontaneous breakdown of the 6His-SPAG1-TPR3 substrate in the absence of protease is checked after 60 minutes at 10° C.

### Test of Breakdown of Polyethylene Terephthalate (PET) by the Scaffolded PETase

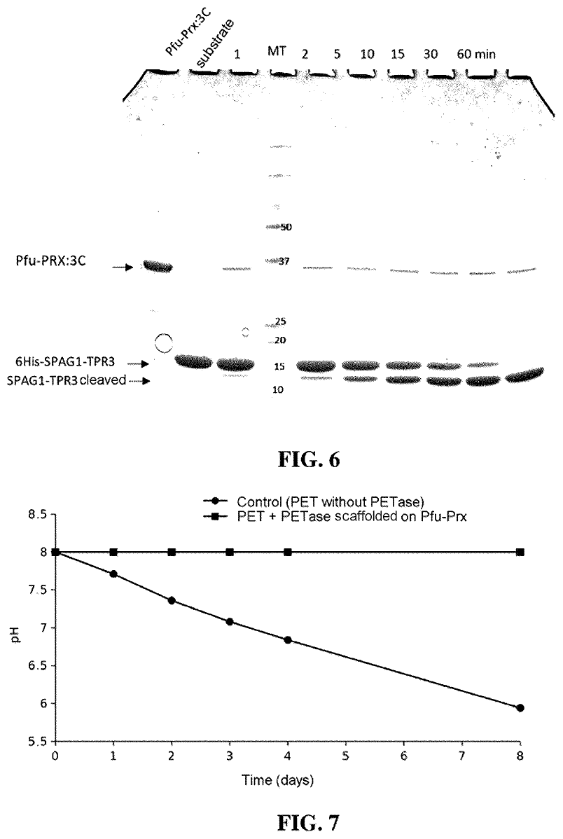

PET film coupons (GoodFellow, 0.25 mm thickness, transparent and amorphous) of approximately 0.5 cm 2 are treated with 30% ethanol for 1 hour at ambient temperature, then dried in the open air. 100 mg of coupons are placed at 50° C. with stirring into a 25 mL conical flask with 5 mL of 100 mM NaPi (pH 8.00) buffer and 1 μM of PETase scaffolded on Pfu-Prx. The reaction is monitored by measuring the pH of the reaction medium, for eight days. A control experiment, without enzyme, is carried out under the same conditions. The results are presented in FIG. 7.

The drop in pH observed within the reaction medium containing the PETase scaffolded on Pfu-Prx is due to the release of terephthalic acid obtained from the breakdown of the PET.

Without enzyme, the pH remains stable. These results indicate that the scaffolded enzyme exhibits hydrolytic activity toward PET.

1-17. (canceled)

18. A method for complexing one or more proteins of interest comprising linking the one or more proteins of interest to one or two N- and/or C-terminal end(s) of one or more monomers of a peroxiredoxin as a scaffold protein, wherein said peroxiredoxin is a multimeric protein from the family of the peroxiredoxins, and wherein said peroxiredoxin has no redox activity.

19. The method of claim 18, characterized in that said one or more proteins of interest are enzymes and the scaffold protein augments the catalytic activity of the enzymes.

20. The method of claim 18, characterized in that said one or more proteins of interest are proteins of the same metabolic pathway and the scaffold protein augments the efficacy of synthesis.

21. The method according to claim 18, characterized in that the peroxiredoxin is the peroxiredoxin TSA1 from Saccharomyces cerevisiae of SEQ ID NO: 1 or the peroxiredoxin from Pyrococcus furiosus of SEQ ID NO: 16.

22. The method according to claim 18, characterized in that the peroxiredoxin is the peroxiredoxin TSA1 comprising a cysteine mutated in position 48 of the sequence SEQ ID NO: 1, in position 171 of the sequence SEQ ID NO: 1 or in positions 48 and 171 of the sequence SEQ ID NO: 1.

23. The method according to claim 18, characterized in that the peroxiredoxin is the peroxiredoxin TSA1 comprising the sequence SEQ ID NO: 29.

24. The method according to claim 18, characterized in that the peroxiredoxin is the peroxiredoxin from Pyrococcus furiosus comprising a cysteine mutated in position 46 of the sequence SEQ ID NO: 16, in position 211 of the sequence SEQ ID NO: 16 or in positions 46 and 211 of the sequence SEQ ID NO: 1.

25. The method according to claim 18, characterized in that the peroxiredoxin is the peroxiredoxin from Pyrococcus furiosus comprising the sequence SEQ ID NO: 52.

26. The method according to claim 18, characterized in that the one or more proteins of interest is fused to one or two N- and C-terminal end(s) of said one or more monomers via a linking sequence.

27. The method according to claim 18, characterized in that said one or more monomers and the one or more proteins of interest are linked via an adapter/peptide ligand pair.

28. A multimeric protein from the family of peroxiredoxins, comprising at least one protein of interest linked to one or two N- and C-terminal end(s) of one or more peroxiredoxin monomers having no redox activity.

29. The multimeric protein according to claim 28 characterized in that said one or more peroxiredoxin monomers and the at least one protein of interest are linked via an adapter/peptide ligand pair.

30. The multimeric protein according to claim 28, comprising at least two different proteins of interest which are linked to one or two N- and C-terminal end(s) of the one or more peroxiredoxin monomers having no redox activity.

31. The multimeric protein according to claim 28, characterized in that the peroxiredoxin is peroxiredoxin TSA1 from Saccharomyces cerevisiae of SEQ ID NO: 1 or the peroxiredoxin from Pyrococcus furiosus of SEQ ID NO: 16.

32. The multimeric protein according to claim 31, characterized in that the peroxiredoxin TSA1 comprises a cysteine mutated in position 48 of SEQ ID NO: 1, in position 171 of SEQ ID NO: 1 or in positions 48 and 171 of SEQ ID NO: 1.

33. The multimeric protein according to claim 31, characterized in that the peroxiredoxin from Pyrococcus furiosus comprises a cysteine mutated in position 46 of SEQ ID NO: 16, in position 211 of SEQ ID NO: 16 or in positions 46 and 211 of SEQ ID NO: 16.

34. One or more nucleotide constructs encoding a multimeric protein according to claim 28.

35. The one or more nucleotide constructions according to claim 34 comprising the sequence SEQ ID NO: 51 encoding a peroxiredoxin TSA1 substituted in positions 48 and 171 by a serine and/or an alanine, respectively, or the sequence SEQ ID NO: 53 encoding the peroxiredoxin from Pyrococcus furiosus substituted in positions 46 and 211 by a serine and/or an alanine, respectively.

36. An expression vector comprising the one or more nucleotide constructs according to claim 34.

37. A host cell comprising a multimeric protein according to claim 28.


SEQUENCE LISTING (55 sequences)

SEQ ID NO: 1 - protein, 196 aa, Saccharomyces cerevisiae
MVAQVQKQAPTFKKTAVVDGVFDEVSLDKYKGKYVVLAFIPLAFTFVCPTEIIAFSEAAKKFEEQGAQVLFASTDSEYSLLAWTNIPRKEGGLGPINIPLLADTNHSLSRDYGVLIEEEGVALRGLFIIDPKGVIRHITINDLPVGRNVDEALRLVEAFQWTDKNGTVLPCNWTPGAATIKPTVEDSKEYFEAANK

SEQ ID NO: 2 - protein, 250 aa, Aeropyrum pernix K1
MPGSIPLIGERFPEMEVTTDHGVIKLPDHYVSQGKWFVLFSHPADFTPVCTTEFVSFARRYEDFQRLGVDLIGLSVDSVFSHIKWKEWIERHIGVRIPFPIIADPQGTVARRLGLLHAESATHTVRGVFIVDARGVIRTMLYYPMELGRLVDEILRIVKALKLGDSLKRAVPADWPNNEIIGEGLIVPPPTTEDQARARMESGQYRCLDWWFCWDTPASRDDVEEARRYLRRAAEKPAKLLYEEARTHLH

SEQ ID NO: 3 - protein, 188 aa, Crithidia fasciculata
MSCGAAKLNHPAPEFDDMALMPNGTFKKVSLSSYKGKYVVLFFYPMDFTFVCPTEIIQFSDDAKRFAEINTEVISCSCDSEYSHLQWTSVDRKKGGLGPMAIPMLADKTKAIARAYGVLDEDSGVAYRGVFIIDPNGKLRQIIINDMPIGRNVEEVIRLVEALQFVEEHGEVCPANWKKGDAKKKEGH

SEQ ID NO: 4 - protein, 235 aa, Chlamydomonas reinhardtii
MAALQSASRSSAVAFSRQARVAPRVAASVARRSLVVRASHAEKPLVGSVAPDFKAQAVFDQEFQEITLSKYRGKYVVLFFYPLDFTFVCPTEITAFSDRYKEFKDINTEVLGVSVDSQFTHLAWIQTDRKEGGLGDLAYPLVADLKKEISKAYGVLTEDGISLRGLFIIDKEGVVQHATINNLAFGRSVDETKRVLQAIQYVQSNPDEVCPAGWKPGDKTMKPDPKGSKEYFSAV

SEQ ID NO: 5 - protein, 266 aa, Arabidopsis thaliana
MASVASSTTLISSPSSRVFPAKSSLSSPSVSFLRTLSSPSASASLRSGFARRSSLSSTSRRSFAVKAQADDLPLVGNKAPDFEAEAVFDQEFIKVKLSDYIGKKYVILFFYPLDFTFVCPTEITAFSDRHSEFEKLNTEVLGVSVDSVFSHLAWVQTDRKSGGLGDLNYPLISDVTKSISKSFGVLIHDQGIALRGLFIIDKEGVIQHSTINNLGIGRSVDETMRTLQALQYIQENPDEVCPAGWKPGEKSMKPDPKLSKEYFSAI

SEQ ID NO: 6 - protein, 226 aa, Leishmania infantum
MLRRLPTSCFLKRSQFRGFAATSPLLNLDYQMYRTATVREAAPQFSGQAVVNGAIKDINMNDYKGKYIVLFFYPMDFTFVCPTEIIAFSDRHADFEKLNTQVVAVSCDSVYSHLAWVNTPRKKGGLGEMHIPVLADKSMEIARDYGVLIEESGIALRGLFIIDKKGILRHSTINDLPVGRNVDEALRVLEAFQYADENGDAIPCGWKPGQPTLDTTKAGEFFEKNM

SEQ ID NO: 7 - protein, 199 aa, Rattus norvegicus
MSSGNAKIGHPAPSFKATAVMPDGQFKDISLSDYKGKYVVFFFYPLDFTFVCPTEIIAFSDRAEEFKKLNCQVIGASVDSHFCHLAWINTPKKQGGLGPMNIPLVSDPKRTIAQDYGVLKADEGISFRGLFIIDDKGILRQITINDLPVGRSVDEILRLVQAFQFTDKHGEVCPAGWKPGSDTIKPDVNKSKEYFSKQK

SEQ ID NO: 8 - protein, 199 aa, Leishmania major
MSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQ

SEQ ID NO: 9 - protein, 271 aa, Homo sapiens
MEALPLLAATTPDHGRHRRLLLLPLLLFLLPAGAVQGWETEERPRTREEECHFYAGGQVYPGEASRVSVADHSLHLSKAKISKPAPYWEGTAVIDGEFKELKLTDYRGKYLVFFFYPLDFTFVCPTEIIAFGDRLEEFRSINTEVVACSVDSQFTHLAWINTPRRQGGLGPIRIPLLSDLTHQISKDYGVYLEDSGHTLRGLFIIDDKGILRQITLNDLPVGRSVDETLRLVQAFQYTDKHGEVCPAGWKPGSETIIPDPAGKLKYFDKLN

SEQ ID NO: 10 - protein, 196 aa, Saccharomyces cerevisiae
MVAEVQKQAPPFKKTAVVDGIFEEISLEKYKGKYVVLAFVPLAFSFVCPTEIVAFSDAAKKFEDQGAQVLFASTDSEYSLLAWTNLPRKDGGLGPVKVPLLADKNHSLSRDYGVLIEKEGIALRGLFIIDPKGIIRHITINDLSVGRNVNEALRLVEGFQWTDKNGTVLPCNWTPGAATIKPDVKDSKEYFKNANN

SEQ ID NO: 11 - protein, 198 aa, Helicobacter pylori
MLVTKLAPDFKAPAVLGNNEVDEHFELSKNLGKNGVILFFWPKDFTFVCPTEIIAFDKRVKDFHEKGFNVIGVSIDSEQVHFAWKNTPVEKGGIGQVSFPMVADITKSISRDYDVLFEEAIALRGAFLIDKNMKVRHAVINDLPLGRNADEMLRMVDALLHFEEHGEVCPAGWRKGDKGMKATHQGVAEYLKENSIKL

SEQ ID NO: 12 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 13 - protein, 187 aa, Salmonella typhimurium
MSLINTKIKPFKNQAFKNGEFIEVTEKDTEGRWSVFFFYPADFTFVCPTELGDVADHYEELQKLGVDVYSVSTDTHFTHKAWHSSSETIAKIKYAMIGDPTGALTRNFDNMREDEGLADRATFVVDPQGIIQAIEVTAEGIGRDASDLLRKIKAAQYVAAHPGEVCPAKWKEGEATLAPSLDLVGKI

SEQ ID NO: 14 - protein, 185 aa, Schistosoma mansoni (Blood fluke)
MVLLPNRPAPEFKGQAVINGEFKEICLKDYRGKYVVLFFYPADFTFVCPTEIIAFSDQVEEFNSRNCQVIACSTDSQYSHLAWDNLDRKSGGLGHMKIPLLADRKQEISKAYGVFDEEDGNAFRGLFIIDPNGILRQITINDKPVGRSVDETLRLLDAFQFVEKHGEVCPVNWKRGQHGIKVNQK

SEQ ID NO: 15 - protein, 199 aa, Trypanosoma cruzi
MSCGDAKLNHPAPDFNETALMPNGTFKKVALTSYKGKWLVLFFYPMDFTFVCPTEICQFSDRVKEFSDIGCEVLACSMDSEYSHLAWTSIERKRGGLGQMNIPILADKTKCIMKSYGVLKEEDGVAYRGLFIIDPKQNLRQITVNDLPVGRDVDEALRLVKAFQFVEKHGEVCPANWKPGDKTMKPDPEKSKEYFGAVA

SEQ ID NO: 16 - protein, 216 aa, Pyrococcus furiosus
MIVIGEKFPEVEVKTTHGVIKLPDHFTKQGKWFMLFSHPADFTPVCTTEFYGLQIRLEKFRELGVEPIGLSVDQVFSHLKWMEWIKEKLGVEIEFPVIADDRGDLAEKLGMIPSGSTITARAVFIVDDKGIIRAIVYYPAEVGRDWDEILRLVKALKISTEKGVALPHKWPNNELIGDKVIVPPASSVEQIKEREEAKAKGEIECYDWWFCYKKLE

SEQ ID NO: 17 - protein, 216 aa, Pyrococcus horikoshii
MVVIGEKFPEVEVKTTHGVIKLPDYFTKQGKWFILFSHPADFTPVCTTEFYGMQKRVEEFRKLGVEPIGLSVDQVFSHIKWIEWIKDNLSVEIDFPVIADDRGELAEKLGMIPSGATITARAVFVVDDKGIIRAIVYYPAEVGRDWDEILRLVKALKISTEKGVALPHKWPNNELIGDKVIVPPASTIEEKKQREEAKAKGEIECYDWWFCYKKLE

SEQ ID NO: 18 - protein, 274 aa, Mus musculus
MEARSKLLDGTTASRRWTRKLVLLLPPLLLFLLRTESLQGLESDERFRTRENECHFYAGGQVYPGEASRVSVADHSLHLSKAKISKPAPYWEGTAVINGEFKELKLTDYRGKYLVFFFYPLDFTFVCPTEIIAFGDRIEEFKSINTEVVACSVDSQFTHLAWINTPRRQGGLGPIRIPLLSDLNHQISKDYGVYLEDSGHTLRGLFIIDDKGVLRQITLNDLPVGRSVDETLRLVQAFQYTDKHGEVCPAGWKPGSETIIPDPAGKLKYFDKLN

SEQ ID NO: 19 - protein, 188 aa, Amphibacillus xylanus
MSLIGTEVQPFRAQAFQSGKDFFEVTEADLKGKWSIVVFYPADFSFVCPTELEDVQKEYAELKKLGVEVYSVSTDTHFVHKAWHENSPAVGSIEYIMIGDPSQTISRQFDVLNEETGLADRGTFIIDPDGVIQAIEINADGIGRDASTLINKVKAAQYVRENPGEVCPAKWEEGGETLKPSLDIVGKI

SEQ ID NO: 20 - protein, 196 aa, Ancylostoma ceylanicum
MSKAFIGKPAPDFATKAVFDGDFVDVKLSDYKGKYVVLFFYPLDFTFVCPTEIIAFSDRFPEFKNLNVAVLACSTDSVFSHLAWINTPRKHGGLGDMKIPVLADTNHQIAKDYGVLKDDEGIAYRGLFIIDPKGILRQITINDLPVGRSVDETLRLVQAFQYTDKHGEVCPAGWTPGKDTIKPAVKESKEYFNKAN

SEQ ID NO: 21 - protein, 187 aa, Enterococcus faecalis
MNLINQKLFDFECDAYHDGEFTRVSTEDILGKWSIFFFYPADFSFVCPTELGDMQEHYAHLQELNCEVYSVSEDSHYVHKAWADATETIGKIKYPMLADPNGQLARFFGVLDEASGMAYRASFIVSPEGDIKSYEINDMGIGRNAEELVRKLEASQFVAEHGDKVCPANWQPGEETIAPSLDLVGKI

SEQ ID NO: 22 - protein, 232 aa, Larimichthys crocea
MEEAAHVKNSQCHNYAGGHVYPGEAFRVPVSDHSLHLSKAKISKPAPQWEGTAVINGEFKELKLSDYRGKYLVFFFYPLDFTFVCPTEIIAFSDRVHEFRAINTEVVACSVDSQFTHLAWIITPRKQGGLGPMKIPLLSDLTHQISKDYGVYLEDQGHTLRGLFIIDEKGVLRQITMNDLPVGRSVDETLRLVQAFQYTDKHGEVCPAGWKPGSDTIIPDPSGKLKYFDKMK

SEQ ID NO: 23 - protein, 215 aa, Sulfolobus islandicus
MSEGRIPLIGEKFPEMEVITTHGKIKLPDDYKGRWFVLFSHPGDFTPVCTTEFYSFSKKYEEFKKLNTELIGLSVDSNISHIEWVMWIEKNLKVEVPFPIIADPMGNVAKRLGMIHAESSTATVRAVFIIDDKGTVRLILYYPMEIGRNIDEILRAIRALQLVDKAGVVTPANWPNNELIGDKVINPAPRTIKDAKMRLGQPFDWWFTYKEVKTT

SEQ ID NO: 24 - protein, 195 aa, Plasmodium vivax
MPTYVGKEAPFFKAEAVFGDNSFGEVNLTQFIGKKYVLLYFYPLDFTFVCPSEIIALDKALDAFHERNVELLGCSVDSKYTHLAWKKTPLAKGGIGNIKHTLLSDITKSISKDYNVLFDDSVSLRAFVLIDMNGIVQHLLVNNLAIGRSVDEILRIIDAIQHHEKYGDVCPANWQKGKVSMKPSEEGVAQYLSTL

SEQ ID NO: 25 - protein, 226 aa, Leishmania braziliensis
MLRRLATKCFQRNVQCRGFAATSPVLNMDYQMYRTATVRDPAPQFSGKAVVDGAIKEINSNDYKGKYIVLFFYPMDFTFVCPTEIIAFSDRYLEFEKLNTQVIAVSCDSEYSHLAWVNTPRKKGGLGEMKIPVLADKSMEIARDYGVLIESAGIALRGLFVIDKKGTLRHSTINDLPVGRNVDEVLRVVEAFQYADENGDAIPCGWTPGKPTLDTKKAGEFFEKNM

SEQ ID NO: 26 - protein, 207 aa, Akkermansia muciniphila
MLLIGKPAPHFSANAVVNGTIVPDFSLDQFKGKKYVILFFYPKDFTFVCPTELIGFQEALGEFDKRDVAVVGCSTDSEFSHWAWVNTPRDQGGIQGVSYPIVSDINKTISADYGVLAGDEEIDEDGNVEVNGELIAYRGLFLIDKDGIVRHQLINDFPLGRSIDEAIRVVDALQHFELYGEVCPLGWHKGEAAMTPSHEGVASYLSK

SEQ ID NO: 27 - protein, 187 aa, Escherichia coli
MSLINTKIKPFKNQAFKNGEFIEITEKDTEGRWSVFFFYPADFTFVCPTELGDVADHYEELQKLGVDVYAVSTDTHFTHKAWHSSSETIAKIKYAMIGDPTGALTRNFDNMREDEGLADRATFVVDPQGIIQAIEVTAEGIGRDASDLLRKIKAAQYVASHPGEVCPAKWKEGEATLAPSLDLVGKI

SEQ ID NO: 28 - protein, 195 aa, MYCOBACTERIUM TUBERCULOSIS H37RV
MPLLTIGDQFPAYQLTALIGGDLSKVDAKQPGDYFTTITSDEHPGKWRVVFFWPKDFTFVCPTEIAAFSKLNDEFEDRDAQILGVSIDSEFAHFQWRAQHNDLKTLPFPMLSDIKRELSQAAGVLNADGVADRVTFIVDPNNEIQFVSATAGSVGRNVDEVLRVLDALQSDELCACNWRKGDPTLDAGELLKASA

SEQ ID NO: 29 - protein, 196 aa, Artificial Sequence
  Synthetic TSA-1 of Sacchoromyces cerevisiae mutant
MVAQVQKQAPTFKKTAVVDGVFDEVSLDKYKGKYVVLAFIPLAFTFVSPTEIIAFSEAAKKFEEQGAQVLFASTDSEYSLLAWTNIPRKEGGLGPINIPLLADTNHSLSRDYGVLIEEEGVALRGLFIIDPKGVIRHITINDLPVGRNVDEALRLVEAFQWTDKNGTVLPANWTPGAATIKPTVEDSKEYFEAANK

SEQ ID NO: 30 - protein, 8 aa, Artificial sequence
  Synthetic Linker
GGGSGGGS

SEQ ID NO: 31 - protein, 12 aa, Artificial sequence
  Synthetic Linker
GGGSGGGSGGGS

SEQ ID NO: 32 - protein, 9 aa, Artificial sequence
  Synthetic motif HSP90C
DASRMEEVD

SEQ ID NO: 33 - protein, 9 aa, Artificial sequence
  Synthetic motif HSP90C
DTSRMEEVD

SEQ ID NO: 34 - protein, 121 aa, Artificial Sequence
  Synthetic motif SPAG1-622742
TFKALKEEGNQCVNDKNYKDALSKYSECLKINNKECAIYTNRALCYLKLCQFEEAKQDCDQALQLADGNVKAFYRRALAHKGLKNYQKSLIDLNKVILLDPSIIEAKMELEEVTRLLNLKD

SEQ ID NO: 35 - protein, 122 aa, Artificial sequence
  Synthetic motif SPAG1-206327opt
DYLATREKEKGNEAFNSGDYEEAVMYYTRSISALPTVVAYNNRAQAYIKLQNWNSAEQDCEKVLELEPGNVKALLRRATAYKHQNKLREAREDLKKVLKVEPDNDLAKKTLSEVERDLKNSE

SEQ ID NO: 36 - protein, 116 aa, Artificial sequence
  Synthetic motif RPAP3-281396
QAISEKDRGNGFFKEGKYERAIECYTRGIAADGANALLPANRAMAYLKIQKYEEAEKDCTQAILLDGSYSKAFARRGTARTFLGKLNEAKQDFETVLLLEPGNKQAVTELSKIKKE

SEQ ID NO: 37 - protein, 111 aa, Artificial sequence
  Synthetic motif Tah1
MSQFEKQKEQGNSLFKQGLYREAVHCYDQLITAQPQNPVGYSNKAMALIKLGEYTQAIQMCQQGLRYTSTAEHVAIRSKLQYRLELAQGAVGSVQIPVVEVDELPEGYDRS

SEQ ID NO: 38 - protein, 9 aa, Artificial sequence
  Synthetic motif HSP70C
SGPTIEEVD

SEQ ID NO: 39 - protein, 126 aa, Artificial Sequence
  Synthetic motif ySNU13
MSAPNPKAFPLADAALTQQILDVVQQAANLRQLKKGANEATKTLNRGISEFIIMAADCEPIEILLHLPLLCEDKNVPYVFVPSRVALGRACGVSRPVIAASITTNDASAIKTQIYAVKDKIETLLI

SEQ ID NO: 40 - protein, 22 aa, Artificial sequence
  Synthetic motif Rsa1-238259
TDEDVKKWREERKKMWLLKISN

SEQ ID NO: 41 - protein, 128 aa, Artificial sequence
  Synthetic motif hSNU13
MTEADVNPKAYPLADAHLTKKLLDLVQQSCNYKQLRKGANEATKTLNRGISEFIVMAADAEPLEIILHLPLLCEDKNVPYVFVRSKQALGRACGVSRPVIACSVTIKEGSQLKQQIQSIQQSIERLLV

SEQ ID NO: 42 - protein, 24 aa, Artificial sequence
  Synthetic motif NUFIP1-233255
TPEEIARWREERRKNYPTLANIER

SEQ ID NO: 43 - protein, 19 aa, Artificial sequence
  Synthetic motif Tah1-93111
SVQIPVVEVDELPEGYDRS

SEQ ID NO: 44 - protein, 87 aa, Artificial sequence
  Synthetic motif Pih1-258344
HEQQEDVPEYEVKMKRFKGAAYKLRILIENKAPNSKPDRFSPSYNFAENILYINGKLSIPLPRDIVVNAADIKIFHIRKERTLYIYI

SEQ ID NO: 45 - protein, 60 aa, Artificial sequence
  Synthetic motif RPAP3-396455
ELIEKGHWDDVFLDSTQRQNVVKPIDNPPHPGSTKPLKKVIIEETGNLIQTIDVPDSTTA

SEQ ID NO: 46 - protein, 92 aa, Artificial sequence
  Synthetic motif PIH1D1-199290
GRAESGPEKPHLNLWLEAPDLLLAEIDLPKLDGALGLSLEIGENRLVMGGPQQLYHLDAYIPLQINSHESKAAFHRKRKQLMVAMPLLLVPS

SEQ ID NO: 47 - protein, 71 aa, Artificial sequence
  Synthetic motif ZNHIT3-85155
DRVSLQNLKNLGESATLRSLLLNPHLRQLMVNLDQGEDKAKLMRAYMQEPLFVEFADCCLGIVEPSQNEES

SEQ ID NO: 48 - protein, 34 aa, Artificial sequence
  Synthetic motif NUFIP1-462495
DIRHERNVILQCVRYIIKKDFFGLDTNSAKSKDV

SEQ ID NO: 49 - protein, 95 aa, Artificial sequence
  Synthetic motif Hit1-70164
MNKTLKTKAFDDIYQNSAELQELLKYNTVKFHLAKVYRILSSTVNDGSSGKMNSDLQKELAVNYLNTLRYGGIHYNEAIEEFCQILLDKLNAVKK

SEQ ID NO: 50 - protein, 36 aa, Artificial sequence
  Synthetic motif Rsa1-317352
FANENSQLLDFIRELGDVGLLEYELSQQEKDVLFGS

SEQ ID NO: 51 - DNA, 591 bp, Artificial sequence
  Synthetic TSA-1 Sacchoromyces cerevisiae sequence mutant
atggttgcacaggttcagaaacaggcaccgaccttcaaaaaaaccgcagttgttgatggtgtgtttgatgaagttagcctggataaatacaaaggcaaatatgttgtgctggcctttattccgctggcatttacctttgttagcccgaccgaaattattgcatttagcgaagcagccaaaaagtttgaagaacagggtgcccaggttctgtttgcaagcaccgatagcgaatatagcctgctggcatggaccaatattccgcgtaaagaaggtggtctgggtccgattaacattccgctgctggcagataccaatcatagcctgagccgtgattatggtgttctgattgaagaagagggcgttgcactgcgtggtctgtttatcattgatccgaaaggtgtgattcgccacattaccattaatgatctgccggttggtcgtaatgttgatgaagccctgcgtctggttgaagcatttcagtggaccgataaaaatggtacagttctgcctgcaaattggacaccgggtgcagcaaccattaaaccgaccgttgaagatagcaaagaatattttgaagccgcaaacaaataa

SEQ ID NO: 52 - protein, 216 aa, Artificial sequence
  Synthetic Prx Pyrococcus furiosus mutant
MIVIGEKFPEVEVKTTHGVIKLPDHFTKQGKWFMLFSHPADFTPVSTTEFYGLQIRLEKFRELGVEPIGLSVDQVFSHLKWMEWIKEKLGVEIEFPVIADDRGDLAEKLGMIPSGSTITARAVFIVDDKGIIRAIVYYPAEVGRDWDEILRLVKALKISTEKGVALPHKWPNNELIGDKVIVPPASSVEQIKEREEAKAKGEIESYDWWFSYKKLE

SEQ ID NO: 53 - DNA, 642 bp, Artificial sequence
  Synthetic Prx Pyrococcus furiosus mutant
atgattgtgatcggcgaaaaatttccggaagtggaagttaaaaccacgcatggtgttattaaactgccggatcattttaccaaacaaggcaaatggtttatgctgtttagccatccggcagattttacaccggttagcaccaccgaattttatggtctgcagattcgtctggaaaaattccgtgaactgggtgttgaaccgattggtctgagcgttgatcaggtttttagtcatctgaaatggatggaatggatcaaagaaaaactgggcgtcgaaattgaatttccggttattgcagatgatcgtggtgatctggcagagaaactgggtatgattccgagcggtagcaccattaccgcacgtgcagtttttattgttgatgacaaaggtattatccgtgccattgtttattatcctgccgaagttggtcgtgattgggatgaaattctgcgtctggttaaagcactgaaaatcagcaccgaaaaaggtgttgcactgccgcataaatggccgaataatgaactgattggcgacaaagttattgttccgcctgcaagcagcgttgagcagattaaagaacgtgaagaggcaaaagccaaaggtgaaattgaaagctatgattggtggttcagctacaaaaaa

SEQ ID NO: 54 - protein, 182 aa, Artificial sequence
  Synthetic Protease 3c
GPNTEFALSLLRKNIMTITTSKGEFTGLGIHDRVCVIPTHAQPGDDVLVNGQKIRVKDKYKLVDPENINLELTVLTLDRNEKFRDIRGFISEDLEGVDATLVVHSNNFTNTILEVGPVTMAGLINLSSTPTNRMIRYDYATKTGQCGGVLCATGKIFGIHVGGNGRQGFSAQLKKQYFVEKQ

SEQ ID NO: 55 - protein, 258 aa, Artificial Sequence
  Synthetic PETase
SNPYQRGPNPTRSALTADGPFSVATYTVSRLSVSGFGGGVIYYPTGTSLTFGGIAMSPGYTADASSLAWLGRRLASHGFVVLVINTNSRFDGPDSRASQLSAALNYLRTSSPSAVRARLDANRLAVAGHSMGGGGTLRIAEQNPSLKAAVPLTPWHTDKTFNTSVPVLIVGAEADTVAPVSQHAIPFYQNLPSTTPKVYVELCNASHIAPNSNNAAISVYTISWMKLWVDNDTRYRQFLCNVNDPALCDFRTNNRHCQ